(19) United States
(12) Patent Application Publication
(10) Pub. No.: US 2025/0057192 A1
(43) Pub. Date: Feb. 20, 2025

(54) Methods and Compositions for Improving the Health of Companion Animals

(71) Applicant: Hill's Pet Nutrition, Inc., Overland Park, KS (US)

(72) Inventors: Matthew JACKSON, Topeka, KS (US); Dennis JEWELL, Lawrence, KS (US)

(73) Assignee: Hill's Pet Nutrition, Inc., Overland Park, KS (US)

(21) Appl. No.: 18/720,263

(22) PCT Filed: Dec. 15, 2022
(86) PCT No.: PCT/US2022/081617

§ 371 Date: Jun. 14, 2024

Related U.S. Application Data
(60) Provisional application No. 63/289,797, filed on Dec. 15, 2021.

(51) Int. Cl. A23K 50/40; A23K 10/30; A23K 20/147; A23K 20/158; A23K 20/163; A23K 20/20

(52) U.S. Cl. CPC A23K 50/40; A23K 10/30; A23K 20/147; A23K 20/158; A23K 20/163; A23K 20/30

(57) ABSTRACT

Described herein are methods for deriving a Health Index for Pets (HIP) from a molecular or health observation, a combination thereof or a derivative thereof, using a computational approach. Related compositions and methods are also described herein.

### BACKGROUND

Companion animal health is a complex goal that has a contribution from both the host and the microbiota community living with it. Thus, the nutritionist presented with the optimum nutrition being that which supports and feeds both the host and its microbiota community. Health is maintained and advanced by enhancing both while health is degraded through disruption of either the host or it's microbiota. An example of health degraded is obesity which is a problematic condition in pets and associated with a host of health problems such as inflammation and metabolic endotoxemia. One cause of obesity is overeating due to a lack of satiety. Treating obesity caused in such a manner has proven difficult. Pets require a significant amount of time to achieve weight and fat loss and transition from an obese body to a leaner and healthier body. A treatment plan that shortens this time period would be highly beneficial to pets and owners, particularly if the treatment improved the general satiety of a pet and secondary conditions associated with obesity such as metabolic endotoxemia and inflammation.

Current research has begun to show that improving the pet-microbiome relationship aids in the improvements of pet obesity and subsequent obesity-related conditions.

Thus, there is a need in the art for a pet food that aids in the management of obesity as in other health conditions by optimizing the amount of fat while maintaining satiety. This also improves obesity-related conditions including metabolic endotoxemia, and reduced inflammation.

### BRIEF SUMMARY

In some embodiments, the present invention provides a metric that can be used to evaluate the health of an animal (e.g. a companion animal).

Some embodiments of the present invention provide a pet food composition comprising a fiber complex comprising NDF and crude fiber. Other embodiments of the present invention provide a pet food composition comprising: an anti-inflammatory component; from about 15 wt. % to about 25 wt. %, of NDF; and from about 10 wt. % to about 20 wt. % crude fiber; wherein the total fiber content of the pet food composition exceeds about 30 wt. %; and wherein the pet food composition improves HIP.

In further embodiments, the NDF is greater than 20% wt. of the composition and/or the crude fiber is about 11% to about 20% wt. of the composition. In some embodiments, the pet food composition comprises about 21% NDF and about 13% crude fiber. Still further embodiments of the present invention comprise about 10% to about 50% protein, about 5% to about 50% fat, and/or about 6% to about 12% moisture.

In some embodiments, the present invention comprises an animal feed and fiber bundle. The fiber bundle may be at a concentration of about 10% weight to about 50% by weight, of the pet food composition. In some embodiments, the animal feed is a commercially prepared pet food. The composition of the present invention may comprise a weight ratio of about 1:1 to about 1:10 of the fiber bundle to animal feed.

Certain embodiments of the present invention may provide a method for preventing or treating obesity in animals by increasing fat loss and satiety as well as decreasing obesity-related conditions including but not limited to, metabolic endotoxemia and inflammation. This method may comprise administering any of the compositions described herein to a pet in need thereof. In some embodiments of the present invention, the food intake of the pet may be restricted.

### BRIEF DESCRIPTION OF THE DRAWINGS

FIG. 1 depicts an exemplary pet food formulation according to one embodiment.

FIG. 2 depicts data demonstrating the time-control of Example 1.

FIG. 3 depicts data demonstrating the decrease of inflammatory arachidonates through both phases of Example 1.

FIG. 4 depicts data demonstrating the improvement in endotoxemic status provided by an exemplary composition of the present invention.

FIG. 5 depicts data demonstrating an increase in anti-inflammatory cytokine IL-10.

FIG. 6 depicts data demonstrating a decrease in pro-inflammatory cytokine MCP-1.

FIG. 7 depicts data demonstrating an increase in satiety hormone pancreatic peptide.

FIG. 8 depicts data demonstrating a decrease in hunger hormone ghrelin.

FIG. 9 depicts data demonstrating a decrease in amount of total weight.

FIG. 10 depicts data demonstrating a decrease in amount of total fat.

FIG. 11 provides a list of circulating hormones and cytokines that may be used to calculate HIP in accordance with certain embodiments of the present invention.

FIG. 12 provides a list of micro RNA that may be used to calculate HIP in accordance with certain embodiments of the present invention.

FIG. 13 provides a list of endpoints that may be used to calculate HIP in accordance with certain embodiments of the present invention.

FIGS. 14A and 14B provide graphs of the probability of a feline having chronic kidney disease at the age of 6 years or 15 years, respectively, according to certain embodiments of the present invention.

FIGS. 15A and 15B provide graphs of the probability of a feline having chronic kidney disease based on the feline's creatinine level being below 1.3 mg/dL or 1.9 mg/dL, respectively, in accordance with certain embodiments of the present invention.

FIGS. 16A-16C provide graphs of the probability of a feline having chronic kidney disease based an assessment of one or more factors according to certain embodiments of the present invention.

FIGS. 17A-17I provide graphs of the probability of a feline at the age of 2 years having chronic kidney disease based on an assessment of one or more factors in accordance with certain embodiments of the present invention.

FIGS. 18A-18E provide graphs showing the correlation for various inputs for the respective biomarkers and the effect on chronic kidney disease according to certain embodiments of the present invention.

FIG. 18F provides a graph showing the determined probability of CKD based on the determined effect of the assessed biomarkers and inputs from FIGS. 18A-18E in accordance with certain embodiments of the present invention.

FIGS. 19A-19D provide graphs showing the correlation for various inputs for the respective biomarkers and the effect on IBD according to certain embodiments of the invention.

FIG. 19E provides a graph showing the determined probability of IBD based on the determined effect of the assessed biomarkers and inputs from FIGS. 19A-19D in accordance with certain embodiments of the present invention.

FIGS. 20A-20D provide graphs showing the correlation for various inputs relating to lethargicness, loose stool, vomiting, and a combination of the foregoing three according to certain embodiments of the present invention.

FIGS. 21A-21D provide graphs showing the correlation for the probability of obesity in felines and various inputs relating to IL-4, SDF-1, MCP-1, and a combination of the foregoing three in accordance with certain embodiments of the invention.

FIG. 22A provides a graph of the probability of CKD according to certain embodiments of the present invention.

FIG. 22B provides a graph of the probability of obesity in accordance with certain embodiments of the invention.

FIG. 22C provides a graph of the distribution of probabilities for the overall pet according to certain embodiments of the present invention.

FIG. 23A provides a graph of the probability of CKD in accordance with certain embodiments of the invention.

FIG. 23B provides a graph of the probability of obesity according to certain embodiments of the present invention.

FIG. 23C provides a graph of the distribution of probabilities for the overall pet in accordance with certain embodiments of the invention.

FIG. 24 depicts the impact that an exemplary composition of the present invention has on HIP.

FIG. 25A depicts probability distributions for individual diseases calculated with biomarkers set to various levels of disease probability.

FIG. 25B depicts probability distributions that are combined using a weighted average with the weights listed in each column, resulting in the overall probability of organ/system disease.

### DETAILED DESCRIPTION

The following description of the preferred embodiment(s) is merely exemplary in nature and is in no way intended to limit the invention. The description of illustrative embodiments is intended to be read in connection with the accompanying drawings, which are to be considered part of the entire written description.

As used herein and in the appended claims, the singular forms “a”, “an”, and “the” include plural references unless the context dictates otherwise. The singular form of any class of the ingredients refers not only to one chemical species within that class, but also to a mixture of those chemical species; for example, the term “protein” in the singular form, may refer to a mixture of compounds each of which is also considered a protein

The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e. farm cats), pet cats, ferrets, birds, reptiles, rabbits, and fish.

The term “fiber”, as used herein, could be used interchangeably with “dietary fiber” or “total dietary fiber” and may refer to soluble fiber, insoluble fiber, or a combination of both. Dietary fiber refers to components of a plant which are resistant to digestion by an animal's digestive enzymes.

As used herein, the phrase “soluble fiber” refers to dietary fiber that attracts water during digestion and slows the rate of nutrient absorption. Soluble fiber is resistant to digestion and absorption in the small intestine and undergo complete or partial fermentation in the large intestine, and is typically found in various plant sources, including oat bran, seeds, beans, and certain fruits and vegetables such as beet pulp, guar gum, chicory root, psyllium, pectin, blueberry, cranberry, squash, apples, oats, beans, citrus, barley and peas. The phrase encompasses any source of soluble fiber suitable for the compositions disclosed herein as would be evident to one of skill in the art.

Insoluble fiber may be supplied by any of a variety of sources, including cellulose, whole wheat products, wheat, oat, corn bran, flax seed, grapes, celery, green beans, cauliflower, potato skins, fruit skins, vegetable skins, peanut hulls, and soy fiber.

Crude fiber includes indigestible components contained in cell walls and cell contents of plants such as grains, e.g., hulls of grains such as wheat, oats, rice, corn, and beans.

Examples of fiber sources may comprise apples, apple pomace, barely, beans, beets, beet pulp, dried beet fiber (sugar removed), beta-glucans, blueberry, cauliflower, carrageenan, reduced starch, celery, cellulose, a-cellulose, carboxymethylcellulose, hemicellulose, chicory root, chitins, citrus, citrus pulp, citrus fiber, citrus pectin, cranberry, corn, corn bran, fiber extracts, fiber derivatives, flax seed, grapes, green beans, guar gum, xanthan gum alginates, gum arabic, gum talha, inulin, lignin, oats, oat fiber, wheat oats pectin, peanut hulls, pecan shells, potato, potato skins, psyllium, squash, whole wheat products, soy, soy fiber, fruit skins, vegetable skins, galactooligosaccharides, gentioligosaccharide xylooligosaccharides, fructooligosaccharides, lactulose, mannanoligosaccharides, polydextrose, pectic oligosaccharide, soy oligosaccharides, oligodextran, trehalose, raffinose, stachyose, oligo derivatives from starch, and/or non-starch polysaccharides.

The term “palatability”, as used herein, encompasses all the various properties of food sensed by animals such as texture, taste and aroma. In certain embodiments, the composition has a palatability equal to that of a control composition.

The term “probiotic” may refer to any microorganism suitable for pet consumption and effective for improving the microbial balance in the pet gastrointestinal tract or for other benefits, such as disease or condition relief or prophylaxis, to the pet.

The term “kibble”, as used herein, includes a particulate pellet like component of animal feeds, such as dog and cat feeds, typically having a moisture, or water, content of less than 12% by weight. Kibbles may range in texture from hard to soft and/or may range in internal structure from expanded to dense. Kibbles may be formed by an extrusion process. In nonlimiting examples, a kibble can be formed from a core and a coating to form a kibble that is coated, also called a coated kibble. It should be understood that when the term “kibble” is used, it can refer to an uncoated kibble or a coated kibble.

In some embodiments, the pet food composition is in the form of a kibble. In other embodiments, the pet food composition is in the form of multi-layer kibble and/or a multi-layer kibble comprising a coating. Further, the coating could comprise a palatant. In certain embodiments, the kibble is formed by extrusion. In other embodiments, the composition is in a form selected from: a loaf, a stew, a “meat and gravy” form, a gruel, shreds with a moisture content greater than 50%”, and a product that could be pushed through a syringe. In another embodiment, the present invention comprises 6% wt. to about 12% wt. moisture.

In some embodiments, the kibble may comprise a binder. In certain embodiments the binder includes but is not limited to any of the following or combinations of the following: monosaccharides such as glucose, fructose, mannose, arabinose; di-and trisaccharides such as sucrose, lactose, maltose, trehalose, lactulose; corn and rice syrup solids; dextrins such as corn, wheat, rice and tapioca dextrins; maltodextrins; starches such as rice, wheat, corn, potato, tapioca starches, or these starches modified by chemical modification; alginates, chitosans; gums such as carrageen, and gum arabic; polyols such as glycerol, sorbitol, mannitol, xylitol, erythritol; esters of polyols such as sucrose esters, polyglycol esters, glycerol esters, polyglycerol esters, sorbitan esters; sorbitol; molasses; honey; gelatins; peptides; proteins and modified proteins such as whey liquid, whey powder, whey concentrate, whey isolate, whey protein isolate, high lactose whey by-product, meat broth solids such as chicken broth, chicken broth solids, soy protein, and egg white.

In certain embodiments, the binder includes but is not limited to a lipid and/or lipid derivative. Lipids can be used in combination with water and/or other binder components. Lipids can include plant fats such as soybean oil, corn oil, rapeseed oil, olive oil, safflower oil, palm oil, coconut oil, palm kernel oil, and partially and fully hydrogenated derivatives thereof; animal fats and partially and fully hydrogenated derivatives thereof; and waxes.

In certain embodiments, the present invention may comprise additional ingredients including but not limited to, additives, minerals, vitamins, sources of carbohydrates, fat, protein, additional fiber, amino acids, carotenoids, antioxidants, fatty acids, glucose mimetics, probiotics, prebiotics, and others.

The pet food composition may contain additives known in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may increase the shelf life of the composition. Suitable examples can include preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches.

Additives for coloring, palatability, and nutritional purposes can include colorants, salts (including but not limited to sodium chloride, potassium citrate, potassium chloride, and other edible salts), vitamins, minerals, and flavoring. The amount of such additives in a composition typically is up to about 5% by weight (on a dry matter basis of the composition). Other additives can include antioxidants, omega-3 fatty acids, omega-6 fatty acids, glucosamine, chondroitin sulfate, vegetable extracts, herbal extracts, etc.

In certain embodiments, the pet food composition comprises vitamins and minerals in amounts required to avoid deficiency and maintain health. These amounts are readily available in the art. The Association of American Feed Control Officials (AAFCO) provides recommended amounts of such ingredients for dogs and cats (see Association of American Feed Control Officials. Official Publication, pp. 126-140 (2003)).

Vitamins could as an example include vitamin A, vitamin B1 (thiamine or related sources such as thiamine mononitrate), vitamin B2 (riboflavin), vitamin B3 (niacin), vitamin B5 (pantothenic acid or related sources such as calcium pantothenate), vitamin B6 (pyridoxine or related sources such as pyridoxine hydrochloride), vitamin B8 (folic acid), vitamin B12, vitamin C (ascorbic acid), vitamin D (such as a vitamin D3 supplements), vitamin E, vitamin H (biotin), vitamin K, acetate, choline and choline related sources such as choline chloride, and inositol.

Minerals and trace elements could as an example include calcium, phosphorus, sodium, potassium, magnesium, copper, zinc, choline, and iron salts. Mineral sources can include, for example, sodium selenite, monosodium phosphate, calcium carbonate, potassium chloride, ferrous sulfate, zinc oxide, manganese sulfate, copper sulfate, manganous oxide, potassium iodide, and/or cobalt carbonate.

The term “carbohydrate” as used herein includes polysaccharides (e.g., starches and dextrins) and sugars (e.g., sucrose, lactose, maltose, glucose, and fructose) that are metabolized for energy when hydrolyzed. Examples of carbohydrates suitable for inclusion in the compositions disclosed herein include but are not limited to, corn, grain sorghum, wheat, barley, and rice.

In certain embodiments, the carbohydrate component comprises a mixture of one or more carbohydrate sources. Examples of carbohydrate or carbohydrate ingredients may comprise cereals, grains, corn, wheat, rice, oats, corn grits, sorghum, grain sorghum/milo, wheat bran, oat bran, amaranth, Durum, and/or semolina.

One skilled in the art could manipulate the texture of the final product by properly balancing carbohydrate sources. For example, short chain polysaccharides tend to be sticky and glucy, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional additives is at least partially dependent on the nutritional requirements for different life stages of animals.

In some embodiments, the present invention may comprise about 10% wt. to about 50% wt. of fat. Sources of fats or fat ingredients, may comprise poultry fat, chicken fat, turkey fat, pork fat, lard, tallow, beef fat, vegetable oils, corn oil, soy oil, cottonseed oil, palm oil, palm kernel oil, linseed oil, canola oil, rapeseed oil, fish oil, menhaden oil, anchovy oil, and/or olestra.

In some embodiments, the present invention may comprise about 15% wt. to about 20% wt. of protein. The term “protein” means a polypeptide, or a peptide, or a polymer of amino acids. The term encompasses naturally occurring and non-naturally occurring (synthetic) polymers and polymers in which artificial chemical mimetics are substituted for one or more amino acids. The term also encompasses fragments, variants, and homologs that have the same or substantially the same properties and perform the same or substantially the same function as the original sequence. The term encompasses polymers of any length, including polymers containing from about 2 to 1000, from 4 to 800, from 6 to 600, and from 8 to 400 amino acids. The term includes amino acid polymers that are synthesized and that are isolated and purified from natural sources. Under some embodiments, the terms “polypeptide”, “peptide” or “protein” are used interchangeably.

Protein may be supplied by any of a variety of sources known by those of ordinary skill in the art including plant sources, animal sources, or both. For example, animal sources may include meat, meat-by products, seafood, dairy, eggs, etc. Meats, for example, may include animal flesh such as poultry fish, and mammals including cattle, pigs, sheep, goats, and the like. Meat by-products may include, for example, lungs, kidneys, brain, livers, stomachs and intestines. Plant protein includes, for example, soybean, corn, rice, cottonseed, and peanuts.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

Examples of amino acids may comprise 1-Tryptophan, Taurine, Histidine, Carnosine, Alanine, Cysteine, Arginine, Methionine, Tryptophan, Lysine, Asparagine, Aspartic acid, Phenylalanine, Valine, Threonine, Isoleucine, Histidine, Leucine, Glycine, Glutamine, Taurine, Tyrosine, Homocysteine, Ornithine, Citruline, Glutamic acid, Proline, Serine and/or betaine. Sources of carotenoids may comprise lutein, astaxanthin, zeaxanthin, bixin, lycopene, and/or beta-carotene. Sources of antioxidant ingredients may comprise tocopherols (vitamin E), vitamin C, vitamin A, lipoic acid, plant-derived materials, carotenoids (described above), selenium, and/or CoQ10 (Co-enzyme Q10).

Examples of fatty acid ingredients may comprise arachidonic acid, alphalinoleic acid, gamma linolenic acid, linoleic acid, eicosapentanoic acid (EPA), docosahexanoic acid (DHA), and/or fish oils as a source of EPA and/or DHA. Sources of glucose mimetics may comprise glucose anti-metabolites including 2-deoxy Dglucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose.

Still other ingredients may include beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, and/or keratolytics.

The probiotic component may comprise any suitable bacteria, yeast, microorganisms, and/or mixtures of any thereof. Various probiotic microorganisms are known in the art. In certain embodiments, the probiotic component may comprise bacteria of the order Lactobacillus; bacteria of the genus Bacillus, Bacteroides, and/or Bifidobacterium; yeast of the order Saccharomycesales including the genus Saccharomyces and Candida; and/or mixtures of any thereof. The probiotic may or may not form a spore.

Non-limiting examples of bacteria of the order Lactobacillus suitable for use herein include the genus Streptococci such as Streptococcus lactis, Streptococcus cremoris, Streptococcus diacetylactis, and/or Streptococcus thermophilus; the genus Enterococcus such as Enterococcus faecium, the family Lactobacillillaceae including the genus Pediococcus (i.e. Pediococcus cerevisiae), the genus Leuconostroc, and the genus Lactobacilli such as Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbrukii, Lactobacillus thermophilus, Lactobacillus fermentii, Lactobacillus salvarius, Lactobacillus reuteri, and/or mixtures of any thereof. Nonlimiting examples of bacteria of the genus Bifidobacteria include Bifidobacterium longum, Bifidobacterium infantis, Bifidobacterium bifidum, Bifidobacterium animalis, and/or Bifidobacterium pseudolongum; and/or mixtures of any thereof.

In certain embodiments, the pet food composition may include polyphenols. In some embodiments, the polyphenol source comprises a phenolic compound selected from ellagic acid; gallic acid; protocatechuic acid; p-hydroxybenzoic acid; catechin; and a combination of two or more thereof. In some embodiments, the polyphenol source comprises pecan shells, or any other component of the pecan nut. In some embodiments, the pecan shell may also be a source of lignin-based fiber. Examples of further sources of polyphenols may comprise tea extract, rosemary extract, rosemarinic acid, coffee extract, pecan shells, caffeic acid, turmeric extract, blueberry extract, grape extract, grapeseed extract, and/or soy extract

The pet food composition may undergo feed analysis using any of the variety of methods known to one skilled in the art. Feed analysis may be done to measure any of the nutritional content listed herein including moisture, protein, fiber, carbohydrate, energy, vitamin, mineral, energy, fat, and ash content.

Protein content may be measured and reported in any of the variety of methods known to one skilled in the art. Protein may be reported as crude protein (CP) to measure both true protein content and non-protein nitrogen. Crude protein content may be further differentiated between degradable intake protein (DIP), undegradable intake protein (UIP) and metabolizeable protein (MP). In certain embodiments, protein content may be differentiated to include heat damaged protein or insoluble crude protein (ICP), adjusted crude protein (ACP), and digestible protein (DP).

Fiber content may be measured and reported in any of the variety of methods known to one skilled in the art. Fiber content may be reported as crude fiber (CF), neutral detergent fiber (NDF), acid detergent fiber (ADF) and/or acid detergent lignin (ADL). Crude fiber is generally known to measure the indigestible portion of plant material found in pet food compositions. ADF measures cellulose and lignin, components of plant cell walls. NDF measures the total material found in plant cell walls and includes hemicellulose in addition to the fiber content measured as ADF. ADL measures only the lignin portion of a plant cell wall.

Energy content may be measured and reported in any of the variety of methods known to one skilled in the art. Energy content may be reported as total digestible nutrient (TDN), net energy (NE), metabolizable energy (ME), relative feed value (RFV), and relative forage quality (RFQ).

The present invention may comprise of a 2:1 ratio of NDF to crude fiber. Dietary fiber may be chosen from a group consisting of buckwheat groats, oats, groats, pecan fiber, oat bran, beets, and cellulose. In certain embodiments, the total dietary fiber is greater than 30%. In further embodiments, the NDF is greater than 20% wt. of the composition and/or the crude fiber is about 11% to about 20% wt. of the composition. In some embodiments, the pet food composition comprises about 21% NDF and about 13% crude fiber.

In certain embodiments of the present invention, the pet food composition may achieve any of the desired effects listed herein through the addition of a “fiber bundle” or “bundle” of ingredients. In certain embodiments, the fiber bundle comprises about 0.1% wt. to about 10% wt. of each ingredient. Ingredients for the bundle may include buckwheat groats, oats, pecan fiber, oat bran, fish oil, beet pulp, cranberry pomace, ginger root, yeast cell walls, pomegranate extract, green tea, and curcumin. In some embodiments, the pet food is comprised of at least 25% wt. of the fiber bundle. In other embodiments, the pet food composition comprises 100% of the fiber bundle. In yet another embodiment, the pet food composition may comprise between 10% and 50% of the fiber bundle.

Certain embodiments of the present invention may provide a method for preventing or treating obesity in animals or obesity-related conditions including but not limited to, metabolic endotoxemia and inflammation. This method may comprise administering any of the compositions described herein to a pet in need thereof. In some embodiments, food intake is calculated based upon intake of calories normalized to metabolic bodyweight.

In some embodiments, the present invention provides a metric that can be used to evaluate the health of an animal (e.g. a companion animal). More specifically, the present inventors have discovered that successful feeding of companion animals (e.g., dogs and cats) involves not only feeding the pet but also feeding the microbiome living within and on it. To address this long-standing need, the present inventors have developed a consolidated health metric (the “Health Index for Pets”-HIP) based on several indicies of health used to evaluate companion animals, as well as the microbiome. “In some embodiments, the HIP may be interchangeably used with the “Wellness Index for Pets” (WIP), the “Diagnostic Index for Pets” (DIP), the “Longevity Index for Pets” (LIP), and the like.”

In some embodiments, the present invention utilizes these learnings to maximize the health of companion animals. Without being bound by theory, the present inventors have discovered that simply improving certain health indicies of a companion animal or the microbiome independently, will not achieve the best possible outcome when the combination is considered. Humans and companion animals exist in a constant state of communication and interface with the microbiome. The HIP takes this communication into account and is designed to achieve the best possible outcome for companion animals through measurements of specific analytes in biological samples (saliva, blood (including whole blood, serum and plasma), urine and feces.

In some embodiments, the HIP may be derived from a sub-index selected from: a differential diagnosis index (for pets, cats, dogs, etc.); an optimal health index; a biological age index; a metabolic health index; a microbiome health index; a mitochondrial health index; a renal health index; an urinary health index; a gastrointestinal health index; a dermal health index; a behavioral health index; and an activity health index, or a combination of two or more of these indexes. In some embodiments, any one of these indexes can be used individually or in combination to maximize the health of a companion animal.

In some embodiments, the data or information used to assess the health status or condition of a companion animal using any one of the indexes described herein is gathered by a wearable or an environmental device. In some embodiments, the wearable device tracks and captures motion-, activity-, behavior-related data from a companion animal and/or physiologic data (e.g. heart rate, transdermal water loss, skin pH, presence of biomolecules). In other embodiments, an environmentally located device tracks/monitors: a) movement patterns associated with acceptable versus malfeasant behavior; b) footstep patterns associated with mobility determents; c) ambient molecules associated with stool odor, tracks litter box pH; and/or d) food rate consumption.

In some embodiments, the present invention provides a pet food composition comprising a pet food ingredient in an amount effective to improve HIP through: a) optimizing circulating markers of bacterial cells Lipoteichoic Acid (LTA) and Lipopolysaccharides (LPS); b) optimizing circulating hormones or cytokines; c) optimizing biochemical markers of physiological function; d) optimizing circulating mRNA; c) optimizing microRNA; f) optimizing metabolomics; and/or g) optimizing markers of health, in a companion animal in need thereof. In other embodiments, the present invention provides a pet food composition wherein the optimization of the HIP is observed. In further embodiments, one or more elements from Tables A-E are combined and/or augmented with other molecular, genetic or health observations. In other embodiments, derivatives of elements or combinations of elements are calculated to create HIP. In some embodiments, elements, combinations or derivatives of elements or combinations are selected by computational processes, including, but not limited to regression models, Bayesian approaches, neural networks (general, adversarial, convolutional), classification algorithms and other computational models.

In some embodiments, the present invention provides pet food compositions and related methods to aid in the management of obesity and obesity-related conditions through, e.g., decreasing gut bacterial inflammatory endotoxin in host circulation and decreasing host inflammation.

Other embodiments provide a method for improving HIP in a companion animal comprising administering an effective amount of any one of the pet food compositions described herein to a companion animal in need thereof.

Still further embodiments provide a method for deriving a Health Index for Pets (HIP) from a molecular observation, a health observation (see below, e.g., Tables A-E), a combination thereof or a derivative thereof, using a computational approach.

Lengthy table referenced here
US20250057192A1-20250220-T00001
Please refer to the end of the specification for access instructions.

Lengthy table referenced here
US20250057192A1-20250220-T00002
Please refer to the end of the specification for access instructions.

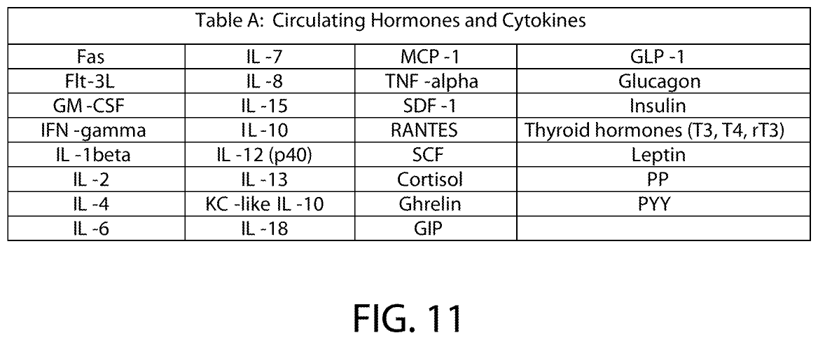

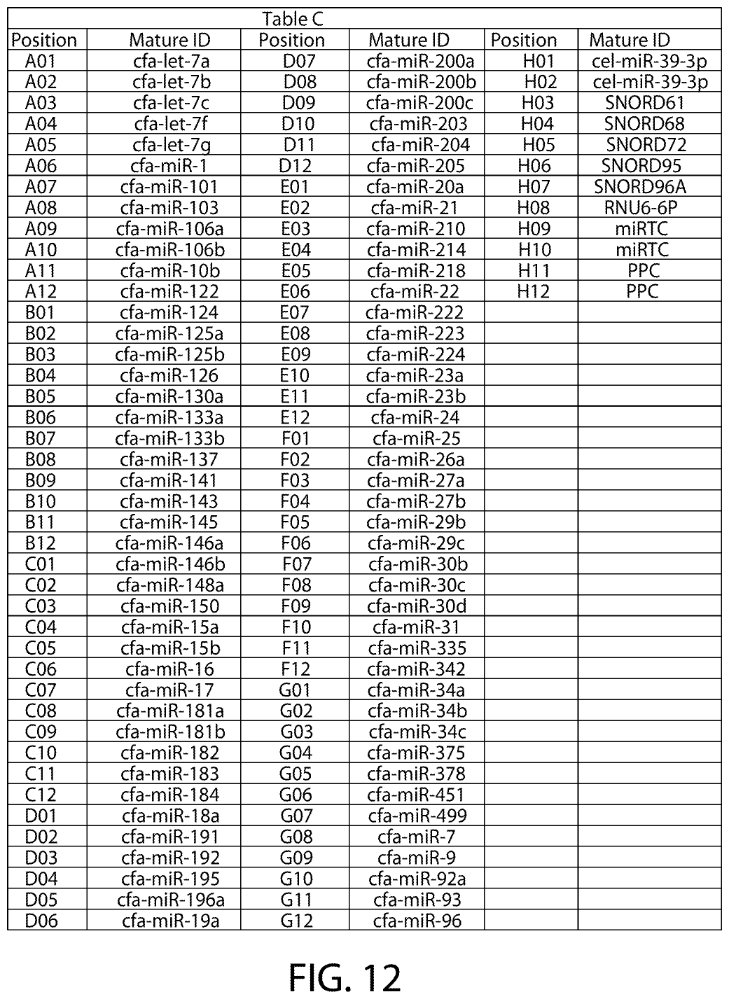

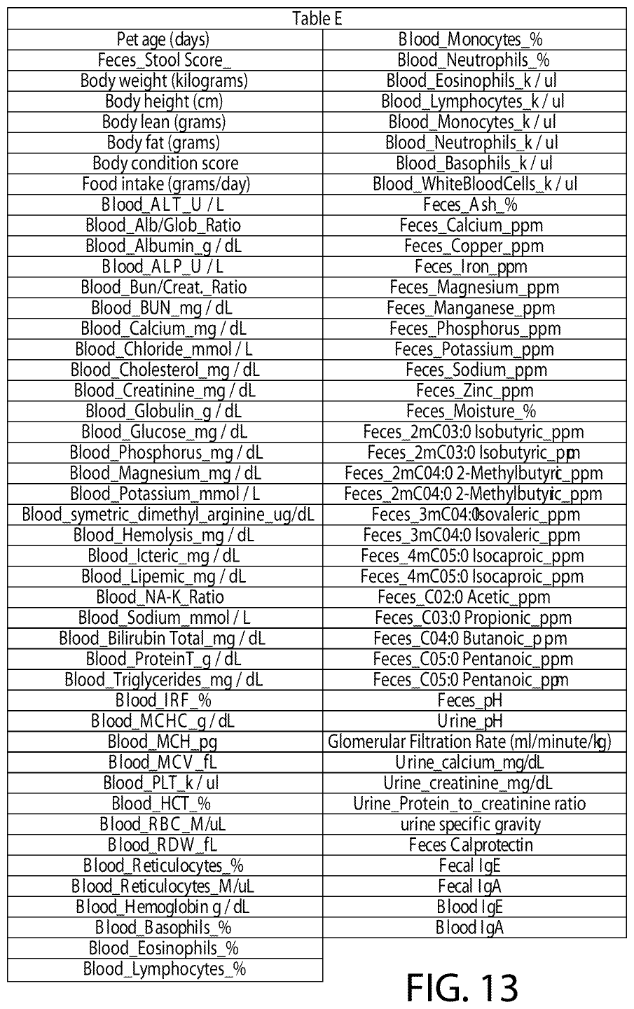

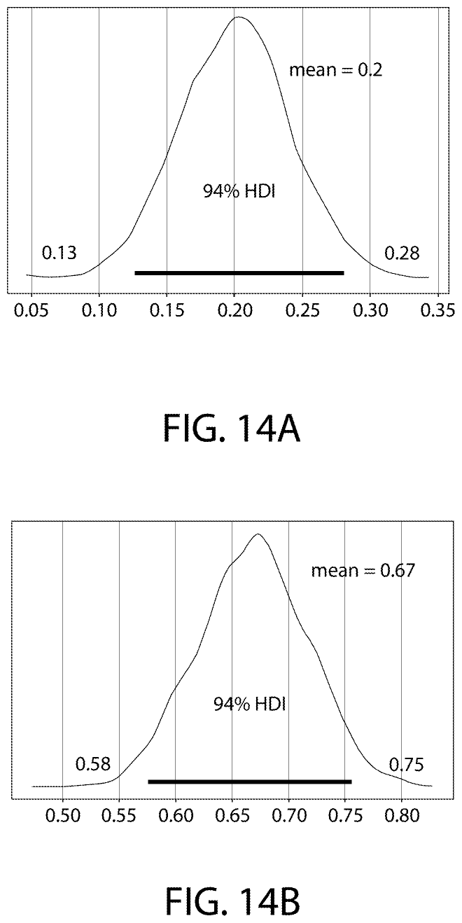

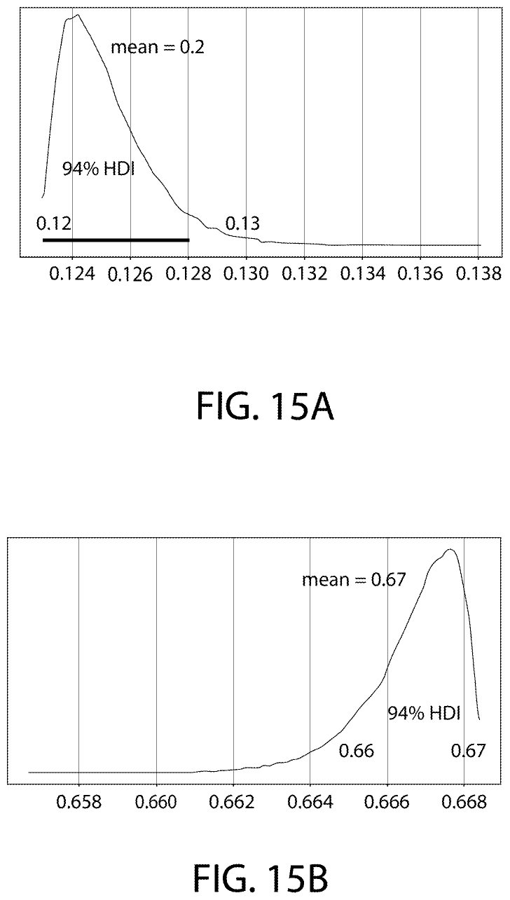

In some embodiments, the circulating hormones are selected from Table A (see, FIG. 11). In some embodiments, the micro RNA is selected from Table C (see, FIG. 12). In some embodiments, the markers of health are selected from Table E (see, FIG. 13).

In some embodiments, the mRNA is selected from Table B (above) and the metabolomics are selected from Table D (above).

In some embodiments, the HIP is independent of specific health indications.

In other embodiments, the HIP is derived from a marker associated with metabolic health, cardiac health, respiratory health, endocrine health, dermatologic health gastrointestinal health, ophthalmic health, behavioral health, emotional health, oral health, renal health, hepatic health, musculoskeletal health, immunologic health, neurologic health, hematologic health and/or reproductive health.

In some embodiments, probabilities can be calculated for any conditions, disorders and diseases that affect metabolic health, cardiac health, respiratory health, endocrine health, dermatologic health gastrointestinal health, ophthalmic health, behavioral health, emotional health, oral health, renal health, hepatic health, musculoskeletal health, immunologic health, neurologic health, hematologic health, reproductive health and/or any listed in The Disease Ontology, a project of the Institute of Genome Sciences.

In some embodiments, the HIP is specific for canines. In other embodiments, the HIP is specific for felines. In yet other embodiments, the HIP is species independent.

Still further embodiments provide a method wherein the computational approach is selected from: a regression model, a Bayesian approach, a neural network (general, adversarial, convolutional), a classification algorithm or a combination thereof.

While other embodiments of the present invention provide methods wherein the HIP is derived from at least one molecular observation identified in Table A, Table B, Table C, Table D or Table E. In other embodiments, the present invention provides a method wherein the HIP is derived from a combination of the molecular observations identified in Table A, Table B, Table C, Table D, Table E. While other embodiments provide a method wherein the molecular observation is augmented with genetic or epigenetic data.

In certain embodiments, the pet food composition is used as a supplement to other pet foods known by one skilled in the art. The pet food composition may be added to another pet food from a 1:1 ratio to a 1:10 ratio. In a preferred embodiment, the present invention is added at a ratio of 1:4.

Some embodiments of the present invention provide a pet food composition comprising:
a component or an ingredient selected from: a. a fiber component comprising neutral detergent fiber (NDF) and crude fiber; b. a macronutrient component (protein intact and hydrolyzed, fat, carbohydrates); c. an optimized mineral component (e.g., controlled calcium, magnesium, phosphorus, sodium, iodine, iron, selenium, or manganese); d. an anti-oxidant component (e.g., vitamin E or vitamin C, a carotenoid including beta-carotene, tocotrienols, glutathione, a botanical, or alpha lipoic acid); e. an anti-inflammatory component (e.g., a biologic ingredient, such as, eggs or quinoa; a fatty acid, an amino acid, or a botanical such as an anthocyanin or a carotenoid); f. an osmolyte manipulator (e.g., betaine); g. a mitochondrial co-factor (e.g. creatine, L-carnitine or acetyl carnitines); h. an amino acid component (essential and/or non-essential amino acids); i. a fatty acid component (essential fatty acids and all saturated, monounsaturated and polyunsaturated fatty acids); j. an anti-anxiety component (e.g., a protein or an isoflavin); k. a physiological process modulator (e.g., glycosaminoglycans); and l. a combination of two or more thereof; wherein a through l is present in an amount effective to improve HIP.

Other embodiments of the present invention provide a pet food composition consisting essentially of: a component or an ingredient selected from: a. a fiber component comprising neutral detergent fiber (NDF) and crude fiber; b. a macronutrient component (protein intact and hydrolyzed, fat, carbohydrates); c. an optimized mineral component (e.g., controlled calcium, magnesium, phosphorus, sodium, iodine, iron, selenium, or manganese); d. an anti-oxidant component (e.g., vitamin E or vitamin C, a carotenoid including beta-carotene, tocotrienols, glutathione, a botanical, or alpha lipoic acid); c. an anti-inflammatory component (e.g., a biologic ingredient, such as, eggs or quinoa; a fatty acid, an amino acid, or a botanical such as an anthocyanin or a carotenoid); f. an osmolyte manipulator (e.g., betaine); g. a mitochondrial co-factor (e.g. creatine, L-carnitine or acetyl carnitines); h. an amino acid component (essential and/or non-essential amino acids); i. a fatty acid component (essential fatty acids and all saturated, monounsaturated and polyunsaturated fatty acids); j. an anti-anxiety component (e.g., a protein or an isoflavin); k. a physiological process modulator (e.g., glycosaminoglycans); and l. a combination of two or more thereof; wherein a through l is present in an amount effective to improve HIP.

In some embodiments, the pet food composition improves HIP by at least about 10%, optionally at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

In further embodiments, the pet food composition provides at least a 2-fold improvement in HIP, optionally a 3-fold improvement in HIP, or a 4-fold improvement in HIP, or a 5-fold improvement in HIP, or a greater than 5-fold improvement in HIP.

Still further embodiments of the present invention provide a method for deriving a Health Index for Pets (HIP) from a molecular or health observation (see, e.g., Tables A-E), a combination thereof or a derivative thereof, using a computational approach. In some embodiments, the HIP is independent of specific health indications.

In some embodiments, the HIP is derived from a marker associated with metabolic health, cardiac health, respiratory health, endocrine health, dermatologic health gastrointestinal health, ophthalmic health, behavioral health, emotional health, oral health, renal health, hepatic health, musculoskeletal health, immunologic health, neurologic health, hematologic health and/or reproductive health.

In other embodiments, the HIP is specific for canines. In further embodiments, the HIP is specific for felines. While in other embodiments, the HIP is species independent.

In some embodiments, the computational approach is selected from: a regression model, a Bayesian approach, a neural network (general, adversarial, convolutional), a classification algorithm or a combination thereof. Still further embodiments of the present invention provide methods wherein the HIP is derived from at least one molecular observation identified in Table A, Table B, Table C, Table D, Table E. In some embodiments, the HIP is derived from a combination of the molecular observations identified in Table A, Table B, Table C, Table D, Table E. Yet other embodiments provide methods wherein the molecular observation is augmented with genetic or epigenetic data.

In some embodiments, the HIP may be referred to as the Holistic Health Index (“HHI”); and the terms may be used interchangeably from time to time.

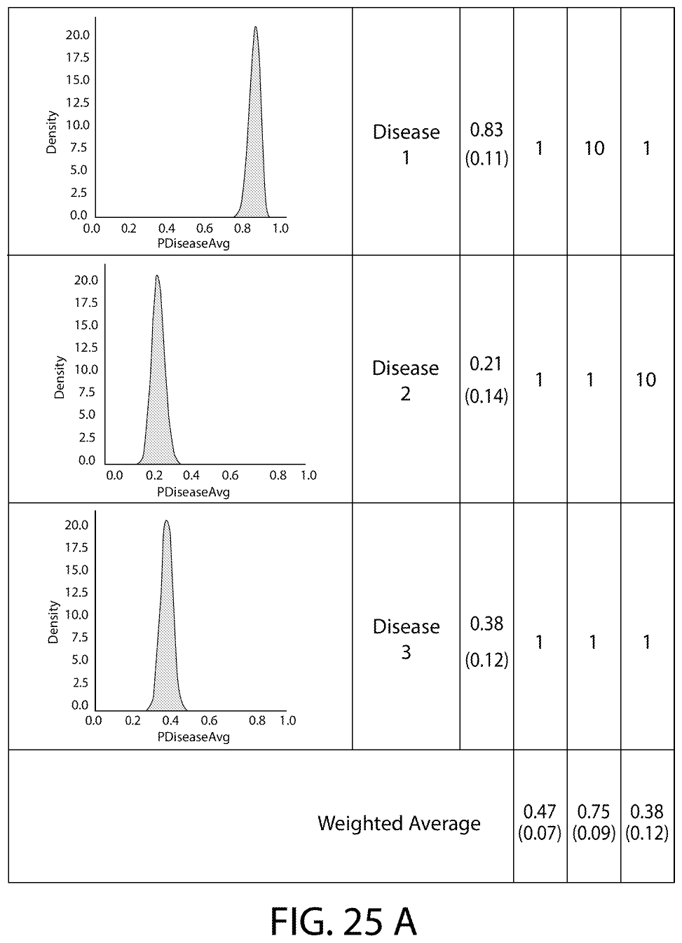

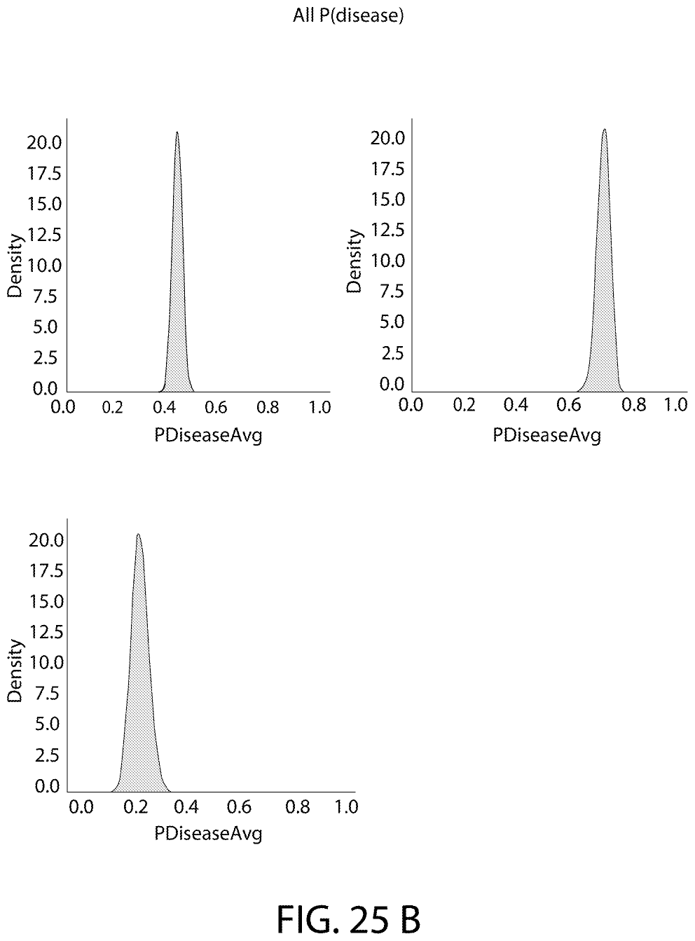

In other embodiments, the present invention provides a method for determining the HIP by combining diseases/conditions that affect various organs/systems. In some embodiments, the HIP is calculated taking an average of the probability distributions for a plurality of diseases/conditions. An example from simulated data of a weighted average combined index illustrated in FIG. 25A and FIG. 25B, wherein the probability distribution for three different diseases were calculated with biomarkers set to various levels of disease probability. The disease probability distributions were then combined using a weighted average. With the weights set to either 1 or 10, the overall probability of organ/system disease is pulled toward the higher weighted distribution.

When combining disease probabilities into organ/system level probabilities, weighting can be applied to each disease probability. These weights can be determined based upon treatability, expert opinion, impact of disease on organ function, quality of life, lifespan or any other aspects of the pets health as well as any combination of these. When combining organ/system health probabilities into whole pet health level probabilities, weighting can be applied to each organ/system health probability. These weights can be determined based upon treatability, expert opinion, impact of organ function on overall health, quality of life, lifespan or any other aspects of the pets health as well as any combination of these.

In some embodiments, the HIP is determined by calculating the aggregate risk of developing one or more diseases or conditions that companion animals are afflicted with.

In some embodiments, the methods and indices of the present invention can be used to communicate the overall health status of a pet for research. In other embodiments, the HIP can be used to communicate the overall health status of a pet for veterinary triage.

In further embodiments, the methods and indices of the present invention can be used to monitor the health of a companion animal with transparency into the factors affecting each aspect of the companion animal's health. In some embodiments, the methods and indices of the present invention can be used to provide personalized nutrition/feeding/activity/veterinary follow up recommendations to pet parents. In other embodiments, the methods and indices of the present invention can be used to assess and/or demonstrate the performance or efficacy of interventions designed to modulate behavior and/or improve health, e.g., pharmaceuticals, exercise, food, visual and auditory stimulations (e.g. lighting, music), and the like. In further embodiments, the methods and indices of the present invention can be used to assess environmental, physical and other factors that negatively impact health or behavior (e.g. noise, allergens, odors, physical exertion, etc).

In some embodiments, the HIP can be calculated by transforming biomarker sample distributions for healthy pets, and then calculating z scores and performing a weighted average across them. In other embodiments, data is grouped and labeled as healthy or diseased and the distance of the indicators from a new pet are used to calculate the HIP.

In yet other embodiments, the HIP is calculated using a graph database to characterize disease likelihood, severity, treatment, side effects, comorbidities and use as deep learning embedding. In some embodiments, the HIP is calculated by creating neural nets for each disease/organ system with input from diagnosis, demographics, standard analysis, signs/symptoms, microbiome, metabolomics to generate scores that are explainable to the level of the disease/organ but not down to the indicator.

In other embodiments, a priority matrix of severity, treatability and quality of life impact is used to bring individual disease/organ indices together. When set up as an optimization problem, recommendations can be made by setting the health index as the objective function. In further embodiments, a collaborative filtering algorithm using a similarity metric between new cases and known disease cases is employed to form an overall index.

In some embodiments, the methods and indices of the present invention provide a quantitative measure of the state of the health of a companion animal at a given point in time.

Still further embodiments of the present invention provide methods and indices that can facilitate the diagnosis and treatment of a companion animal.

Embodiments of the present invention will now be further described by way of the following, non-limiting, example.

### EXAMPLES

### Example 1

Diets were formulated according to AAFCO (American Association of Feed Control Officials) and NRC (National Research Council) nutrition recommendations. The finished kibble was produced by extrusion, dried, and coated with palatants. All diets were canine maintenance formulations.

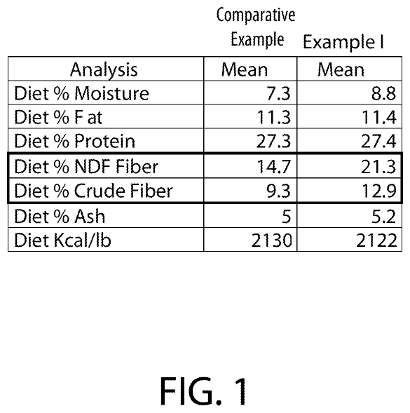

Referring to FIG. 1, the control diet (hereinafter “Comparative Example”) contained only the components of the nutrition recommendations. The inventive diet (hereinafter “Example I”) contained an increased level of NDF and an increased level of crude fiber as compared to the Comparative Example.

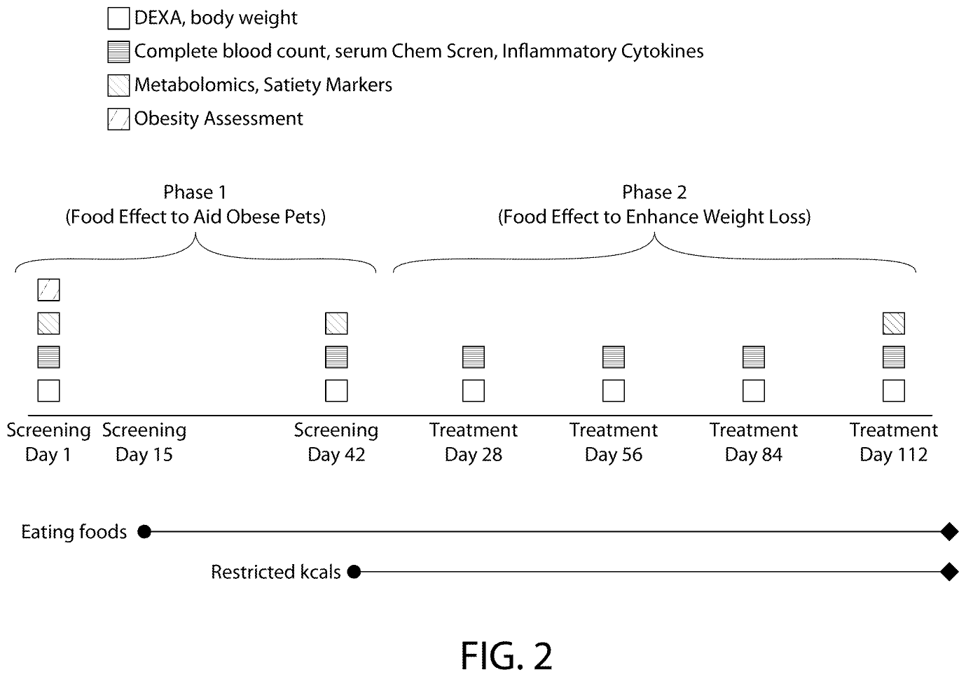

An IACUC approved clinical dietary intervention protocol was implemented which enrolled healthy canine subjects randomized to two groups based on age, weight, obesity status, and sex. The study was a caretaker-blinded, longitudinal design in two phases (see FIG. 2) conducted over about 150 days. Phase 1 consisted of baseline readings and the pets beginning a diet of the control or invention. Phase 2 consisted of the pets continuing the two diets and further, having their calories restricted.

Body composition and subsequent obesity status of each dog in the trial was assessed using duel-energy X-ray absorptiometry (DEXA). Blood draws for satiety hormones (ghrelin, pancreatic peptide), lipopolysaccharide (LPS), cytokines (IL-10 and MCP-1) and metabolomic assessment (proinflammaotry arachidonic acid metabolites) were performed immediately prior to DEXA analysis. Serum was lyophilized and extracted with methanol: water to liberate metabolites from serum matrix.

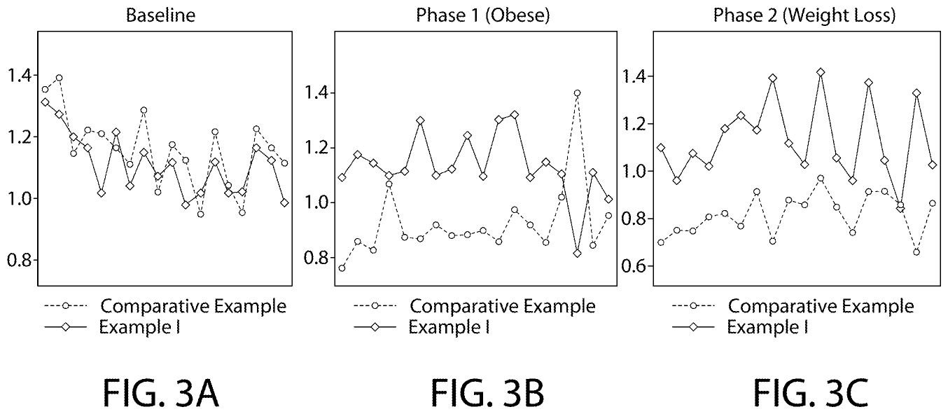

Metabolomics were performed by liquid chromatography-mass spectrometry (LS-MC) with relative fold quantitation. Circulating satiety hormones, LPS and cytokines were assessed by enzyme linked immunosorbent assay (ELISA) and expressed in expressed in picograms per milliliter (pg/ml). The total amount of weight lost and fat lost were calculated through DEXA analysis and expressed in kilograms (kg) and grams (g) respectively. Results are presented in FIGS. 3-9 and depict the relative levels of a given metabolite that was circulating in the dogs after being fed either a control or inventive pet food.

As shown in FIG. 3, an exemplary composition of the present invention decreased small molecule precursors of the prostaglandin pathway. This pathway composed of bound and circulating forms of arachidonic acid contains the precursors of inflammatory prostaglandins produced by cyclooxygenase enzymes. At baseline before feeding there was no significant difference in the arachidonate pathway between the Comparative Example and Example I dogs (see, FIG. 3A). However, once the dogs began consuming the foods, a decrease in the levels of circulating pro-inflammatory arachidonic pathway metabolites was observed in the dogs fed an exemplary composition of the present invention, even before Phase 2 of the study (caloric restriction) was initiated (see, FIG. 3B). These benefits were also readily apparent after Phase 2 was completed (see, FIG. 3C). These results demonstrate that compositions of the present invention promote healthy weight management.

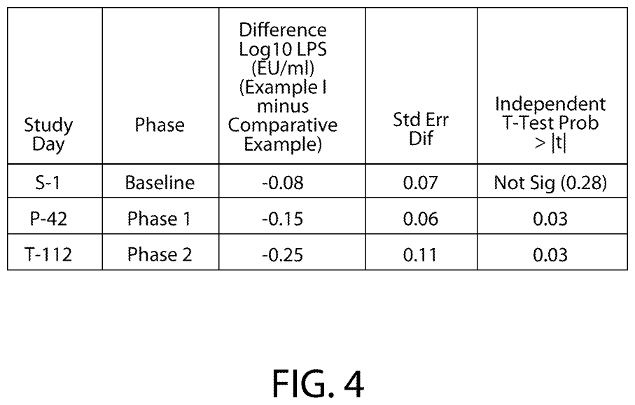

As the data described in FIG. 4 demonstrates, in Phase 1, before calorie restriction, an exemplary composition of the present invention decreased circulating bacterial lipopolysaccharide (LPS). Remarkably, this benefit was still present to a greater extent when compared to a comparative composition, after weight loss (Phase 2).

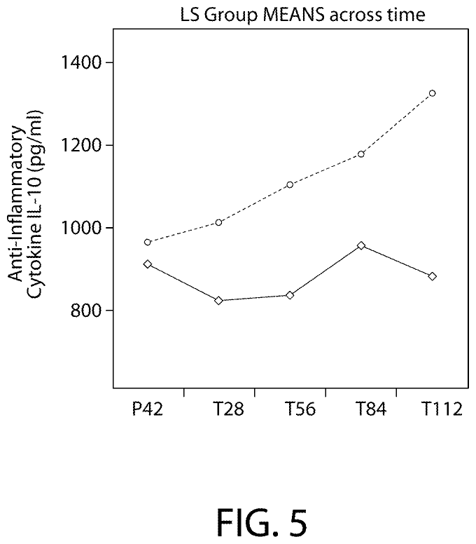

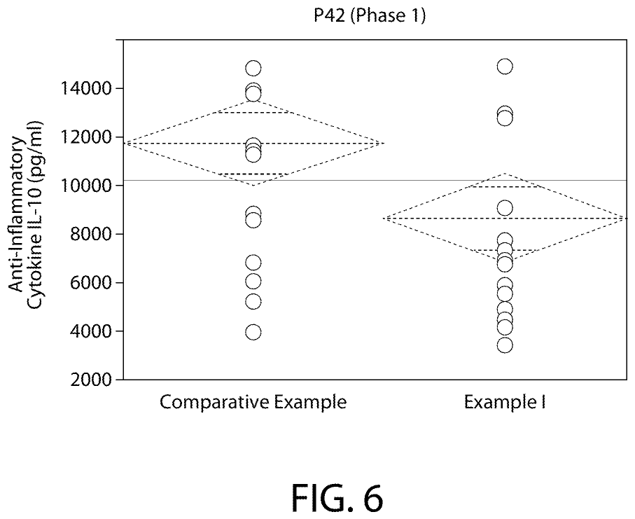

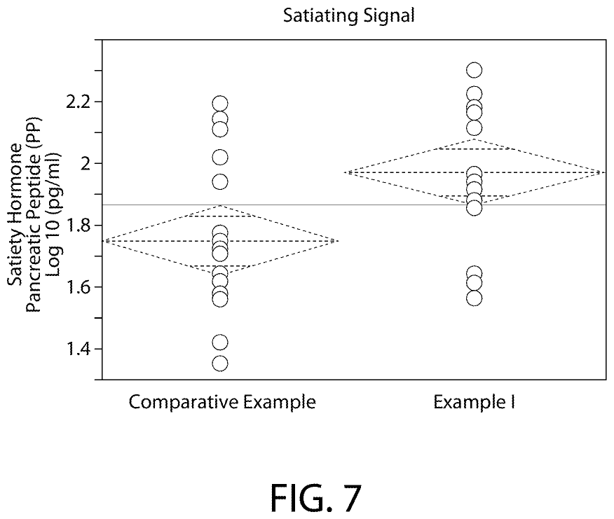

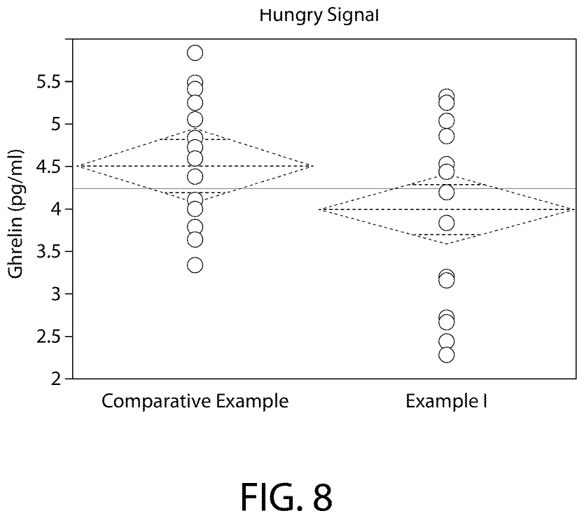

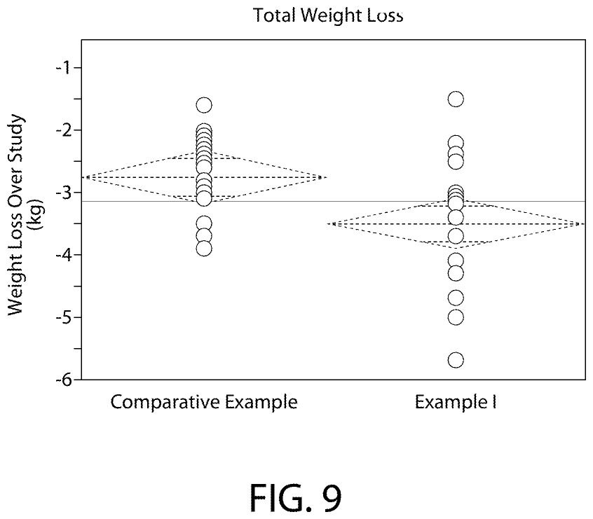

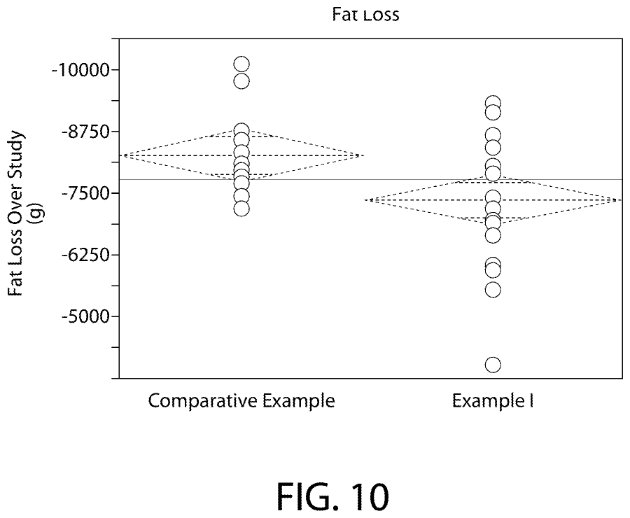

In addition to the dogs fed the invention losing a greater amount of total weight (see FIG. 9) and total fat (see FIG. 10) than the dogs fed the control, they also had a surprisingly greater reduction in obesity-related conditions. As depicted in FIG. 2, this benefit occurred throughout the entire trial during both phases. The invention significantly increased the amount of anti-inflammatory cytokine IL-10 (see, FIG. 5) and significantly reduced the amount of pro-inflammatory cytokine MCP-1 (see FIG. 6). In additional, the dogs fed the invention had greater satiety (see FIG. 7) and lower circulating hunger hormones (see FIG. 8).

### Example 2

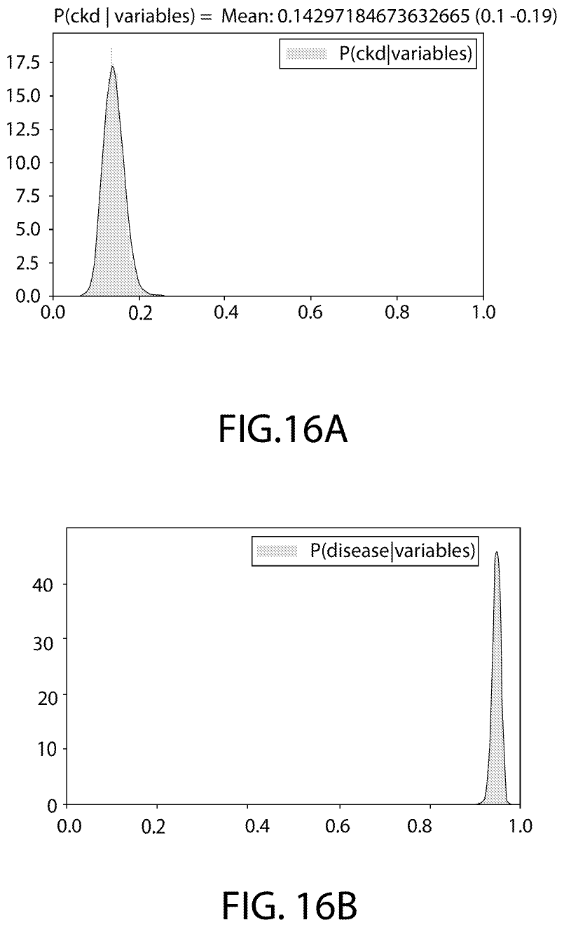

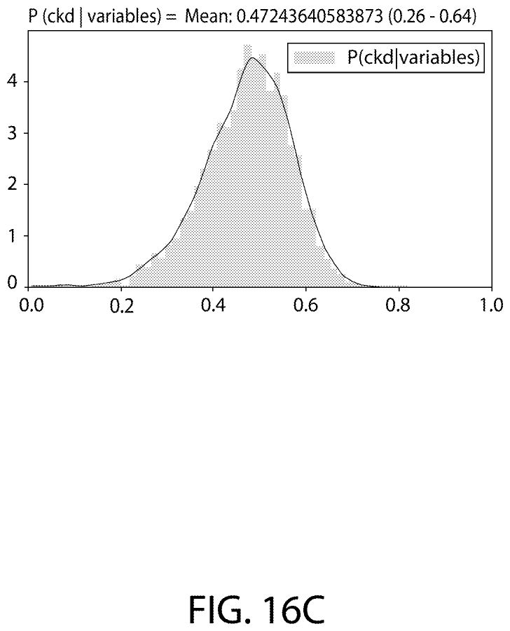

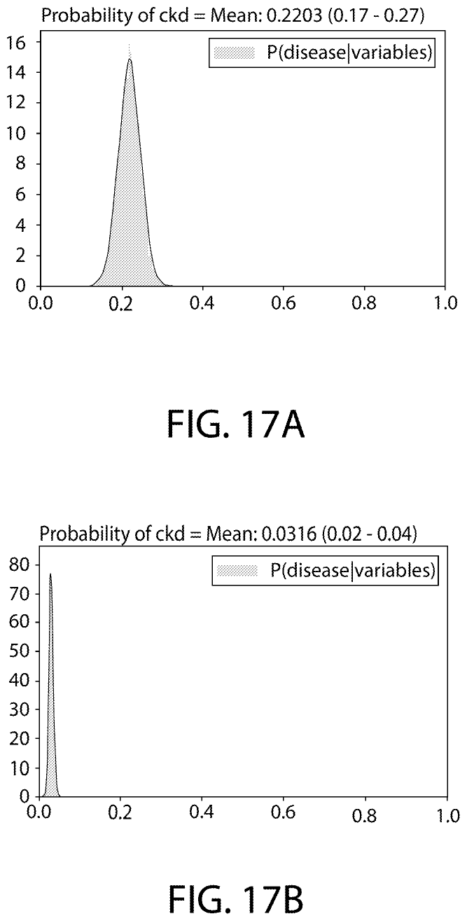

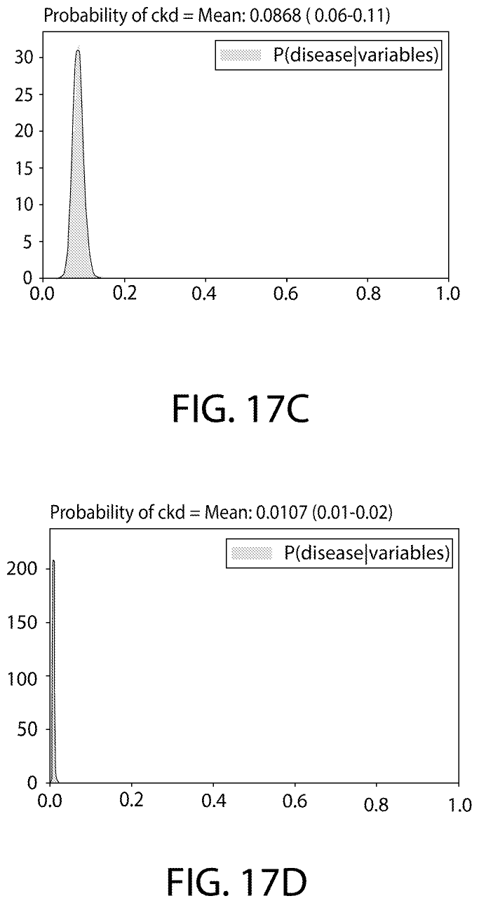

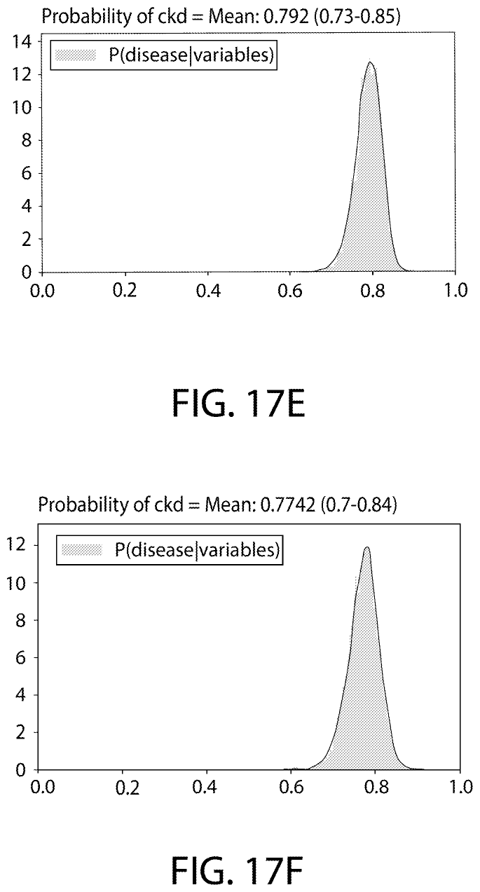

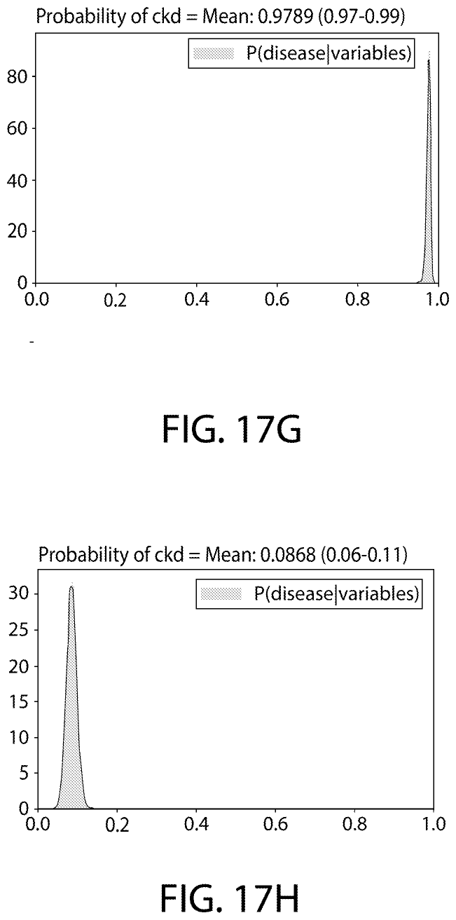

An exemplary, non-limiting index (hereafter “Holistic Health Index”) was prepared in accordance with aspects of the invention. The Holistic Health Index was configured as a health index for companion animals based on inputs relating to health factors for certain organs and biological systems. In this Example, the health factors for certain organs and biological systems were assessed based on biomarkers and certain inputs. Specifically, the health factors were assessed using correlations between the certain inputs and the biomarkers. FIGS. 16A-16C and 17A-17I provide graphs showing the correlation of various inputs for certain biomarkers and the effect on the health factor. The health factors were then combined using Bayes' Rule (Formula I). As seen below, Bayes' Rule was implemented to evaluate the probability of chronic kidney disease based on the biomarkers of creatinine value and feline age and the specific input for the respective feline (Formula II).

$$( I )$$

$$P ( ckd \vert {∧}_{i = 1}^{n} {biomaker}_{i} ) = \frac{P ( ckd ) \prod P ( {biomaker}_{i} \vert ckd )}{\begin{matrix}P ( ckd ) \prod P ( {biomaker}_{i} \vert ckd ) + \\ P ( health ) \prod P ( {biomaker}_{i} \vert health )\end{matrix}}$$

$$( II )$$

$$P ( ckd \vert age , creat ) = \frac{P ( ckd ) P ( age \vert ckd ) P ( creat \vert ckd )}{\begin{matrix}P ( ckd ) P ( age \vert ckd ) P ( creat \vert ckd ) + \\ P ( health ) P ( age \vert health ) P ( creat \vert health )\end{matrix}}$$

For Formula (II), conditional independence of the biomarkers was assumed for combining the determined health factors of the evaluated biomarkers to determine an overall probability of disease given the specific biomarkers. The numerator, which focuses on the probability of the biomarker level given chronic kidney disease is positive, is repeated in the denominator and added to another term which focuses on the probability of the biomarker level given chronic kidney disease is negative.

To assess health factors for multiple organs and/or biological systems, the probability of disease given biomarkers, demographics, and symptoms were assessed and then used for calculating the probability of an unhealthy organ given the specific diseases. Based on the probability of an unhealthy organ, it is possible to determine the probability of an unhealthy pet given the determined probability of diseased organs. The determination of the probability of an unhealthy pet and/or unhealthy organs can be used to also determine the level of health for the feline. The following calculations were also performed:

$$( III )$$

$$P ( ckd \vert {∧}_{i = 1}^{n} {biomaker}_{i} ) = \frac{P ( ckd ) \prod P ( {biomaker}_{i} \vert ckd )}{\begin{matrix}P ( ckd ) \prod P ( {biomaker}_{i} \vert ckd ) + \\ P ( health ) \prod P ( {biomaker}_{i} \vert health )\end{matrix}}$$

Formula (IV), below, relates to the probability of overall disease given the probability of diseased organs.

$$( IV )$$

$$P ( disease {∧}_{i = 1}^{n} {organ}_{i} ) = \frac{P ( disease ) \prod P ( {organ}_{i} \vert disease )}{\begin{matrix}P ( disease ) \prod P ( {organ}_{i} \vert disease ) + \\ P ( health ) \prod P ( {organ}_{i} \vert health )\end{matrix}}$$

Formula (V), below, relates to the probability of health given the probability of healthy organs.

$$( V )$$

$$P ( health \vert {∧}_{i = 1}^{n} {organ}_{i} ) = \frac{P ( health ) \prod P ( {organ}_{i} \vert health )}{\begin{matrix}P ( health ) \prod P ( {organ}_{i} \vert health ) + \\ P ( disease ) \prod P ( {organ}_{i} \vert disease )\end{matrix}}$$

The Holistic Health Index may be adapted to prospectively provide a quantitative measure of the state of the health of the animal at a point in time; communicate uncertainty given the amount of information that we have about the pet; and/or display decline or improvement of health state that would track with a vet assessment.

### Example 3

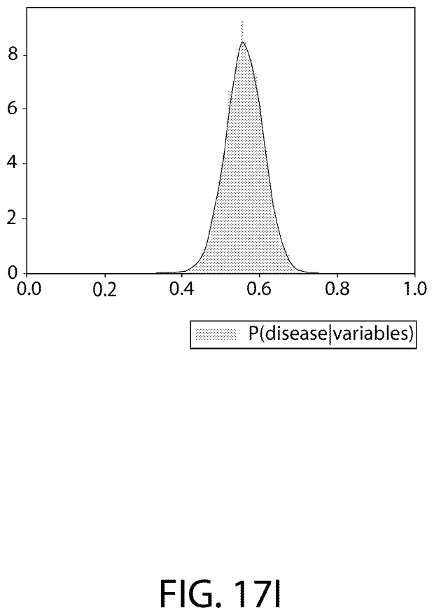

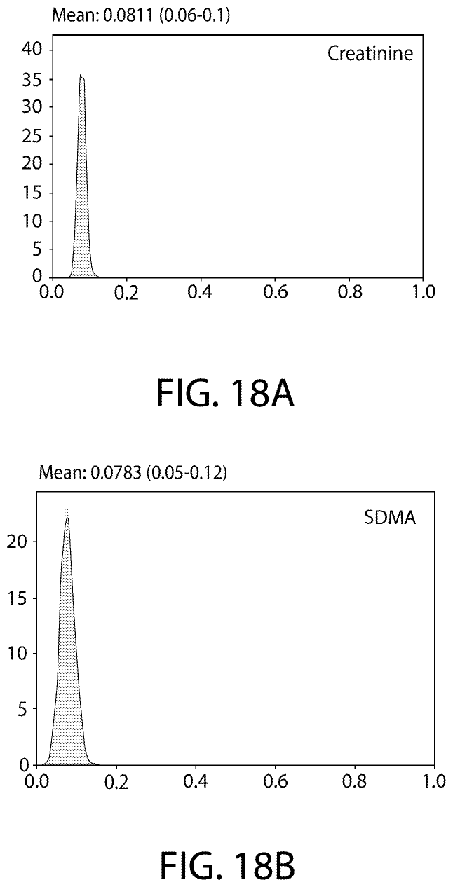

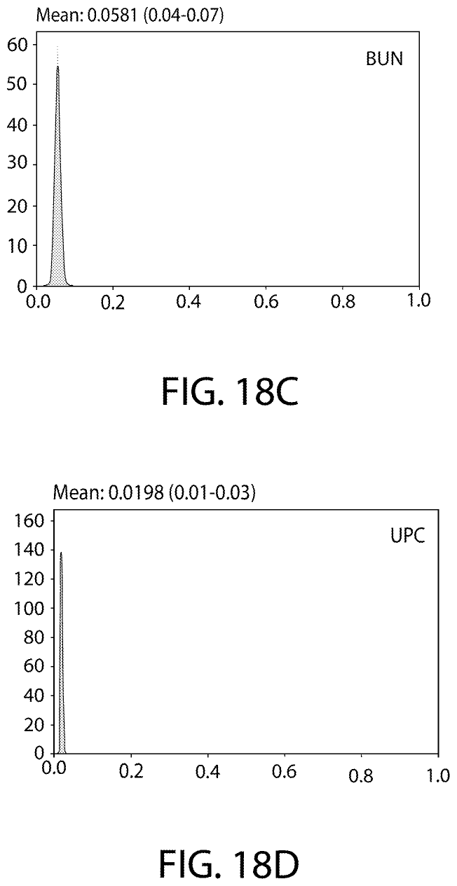

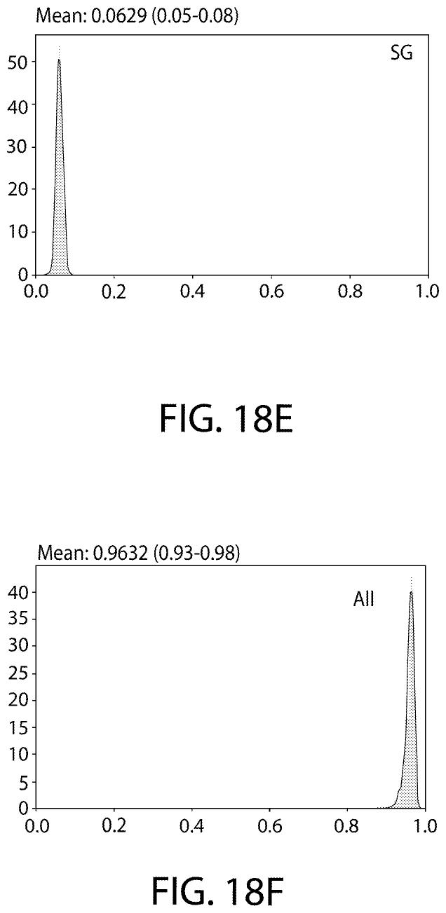

The probability of chronic kidney disease (“CKD”) for felines was assessed using inputs for certain biomarkers and calculating the probability of CKD using the formulas discussed in Example 2. In this Example, the evaluated biomarkers included creatinine, SDMA, BUN, UPC, and SG, with all biomarkers being set to CKD positive. FIGS. 18A-18E provide graphs showing the correlation for various inputs for the respective biomarkers and the effect on the probability of CKD. FIG. 18F provides a graph showing the determined probability of CKD based on the determined effect of the assessed biomarkers and inputs from FIGS. 18A-18E. Table 1 shows the determined probabilities of CKD based on the biomarkers evaluated in FIGS. 18A-18F.

TABLE 1

| Variable | Value | P(ckd) | P(ckd) Range |
|---|---|---|---|
| Creatinine | >1.7 | 0.081 | 0.06-0.1 |
| SDMA | >18 | 0.078 | 0.05-0.12 |
| BUN | >27 | 0.058 | 0.04-0.07 |
| UPC | >0.2 | 0.019 | 0.01-0.03 |
| Specific Gravity | <1.036 | 0.063 | 0.05-0.08 |
| All | | 0.963 | 0.93-0.98 |

### Example 4

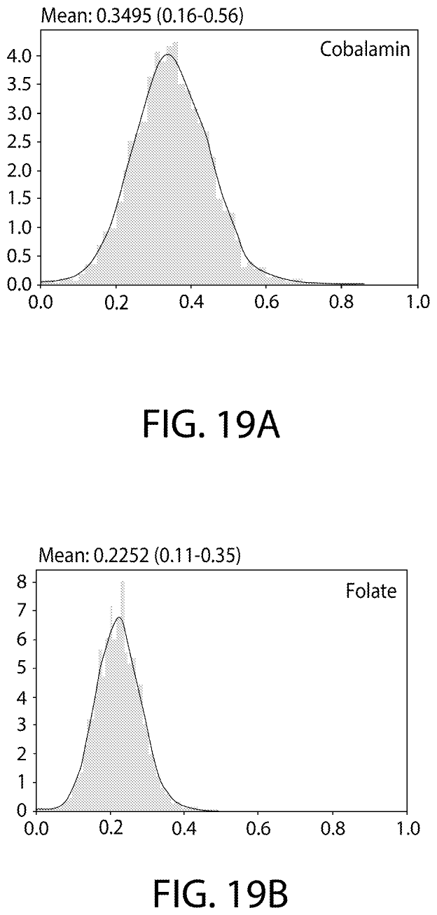

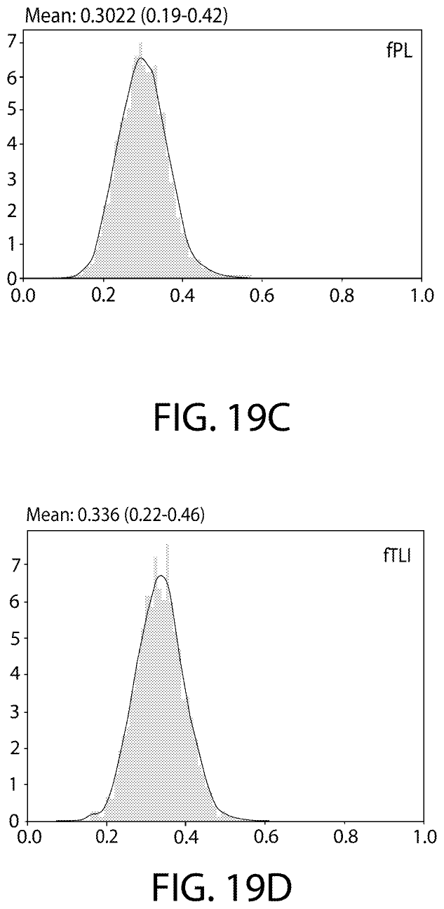

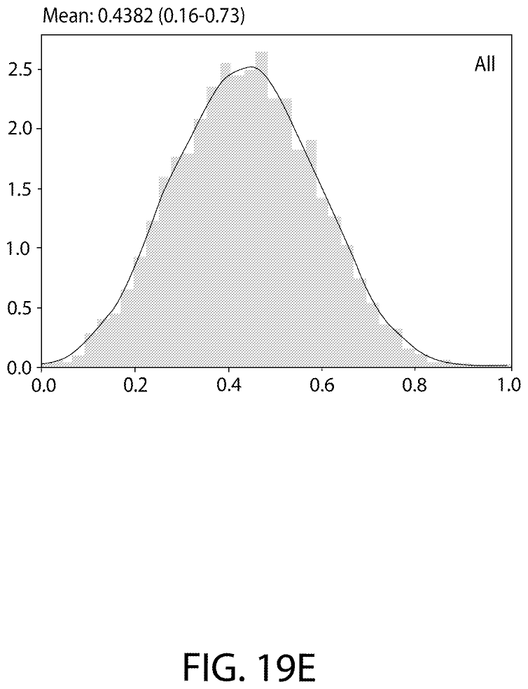

The probability of IBD for felines was assessed using inputs for certain biomarker and calculating the probability of IBD using the formulas discussed in Example 2. In this Example, the evaluated biomarkers included cobalamin, Folate, fPL, fTLI, with all biomarkers being set to IBD positive. FIGS. 19A-19D provide graphs showing the correlation for various inputs for the respective biomarkers and the effect on the probability of IBD. FIG. 19E provides a graph showing the determined probability of IBD based on the determined effect of the assessed biomarkers and inputs from FIGS. 19A-19D. Table 2 shows the determined probabilities of IBD based on the biomarkers evaluated in FIGS. 19A-19E.

TABLE 2

| Variable | Value | P(ibd) | P(ibd) Range |
|---|---|---|---|
| Cobalamin | <290 | 0.35 | 0.16-0.56 |
| Folate | <14 | 0.225 | 0.11-0.35 |
| fPL | >5.4 | 0.302 | 0.19-0.42 |
| fTLI | >60 | 0.336 | 0.22-0.46 |
| All | | 0.438 | 0.16-0.73 |

### Example 5

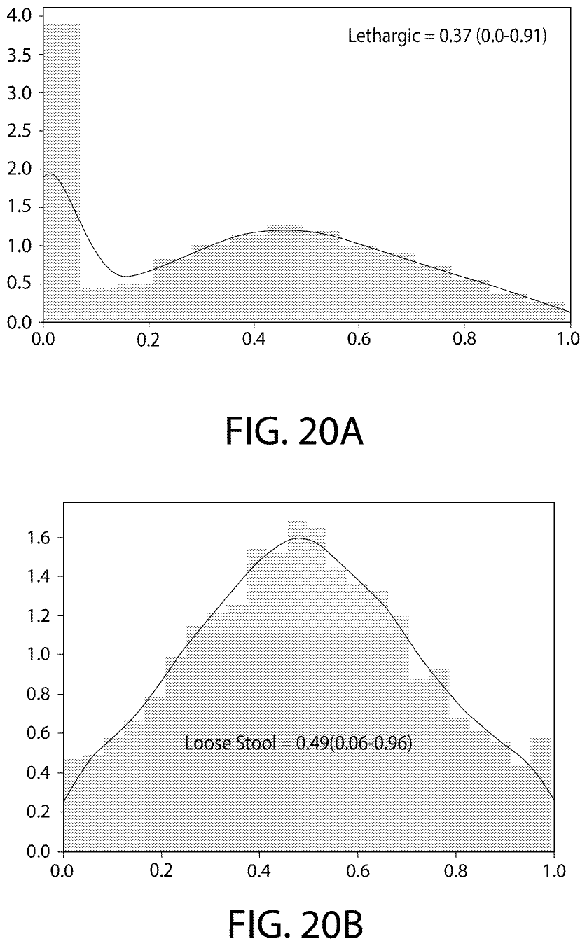

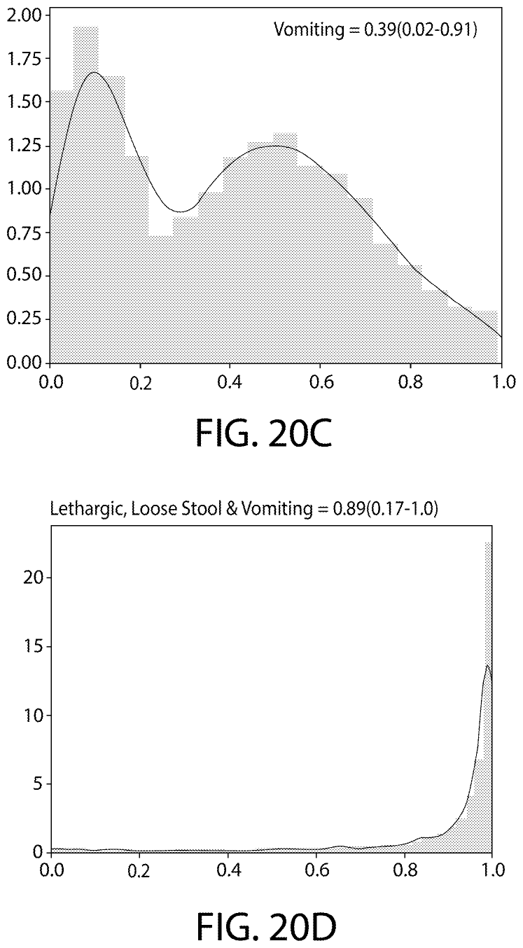

The probability of IBD for canines was assessed using procedures and techniques similar to those discussed in Examples 2-4. In this Example, the probability of IBD was evaluated based on correlations associated with a canine being lethargic, having lose stool, vomiting, and a combination the foregoing three. FIGS. 20A-20D provide graphs showing the correlation for various inputs relating to lethargicness, loose stool, vomiting, and a combination of the foregoing three.

### Example 6

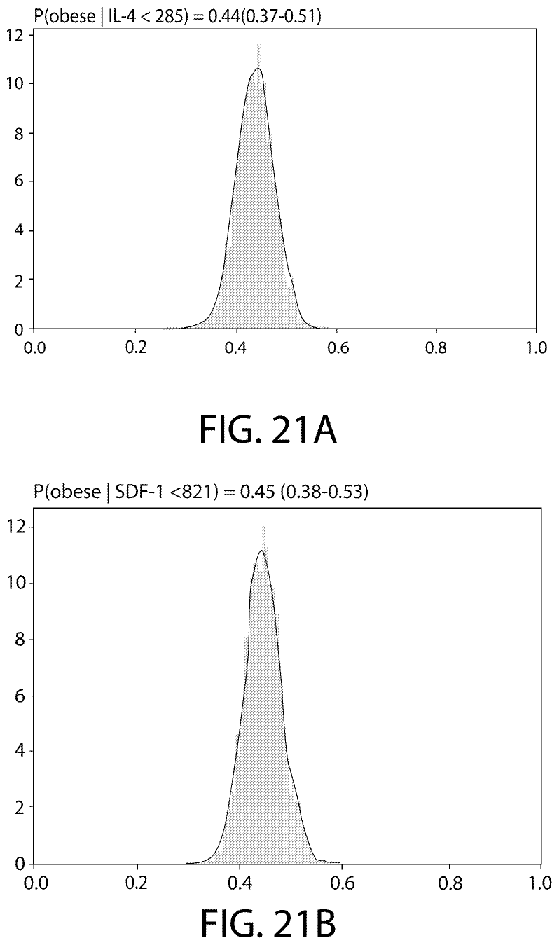

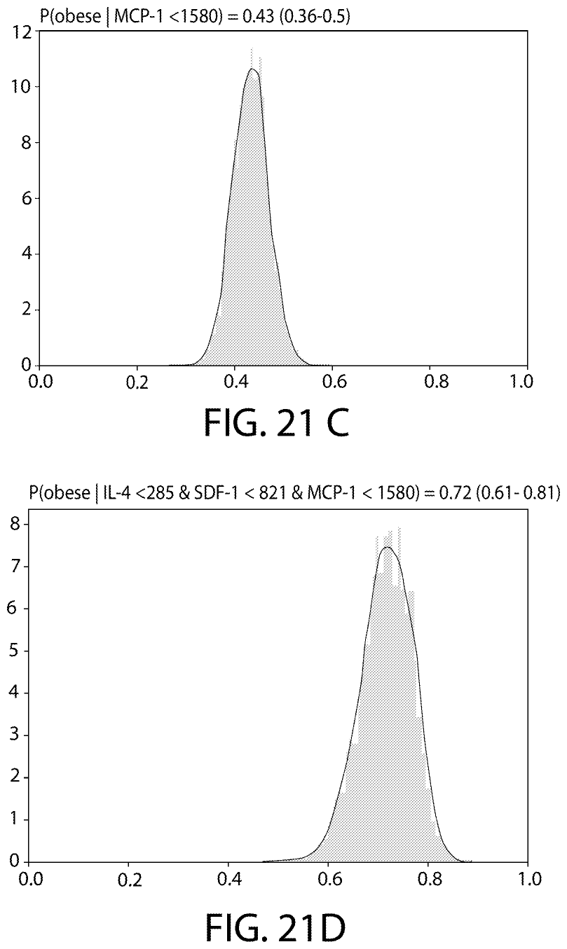

The probability of obesity for felines was assessed using procedures and techniques similar to those discussed in Examples 2-4. In this Example, the probability of obesity was evaluated based on correlations associated with IL-4, SDF-1, MCP-1, and a combination of the foregoing three. Obesity in this Example was defined as DEXA % Fat of greater than 30%. FIGS. 21A-21D provide graphs showing the correlation between the probability of obesity for felines and the various inputs relating to IL-4, SDF-1, MCP-1, and a combination of the IL-4, SDF-1, and MCP-1.

### Example 7

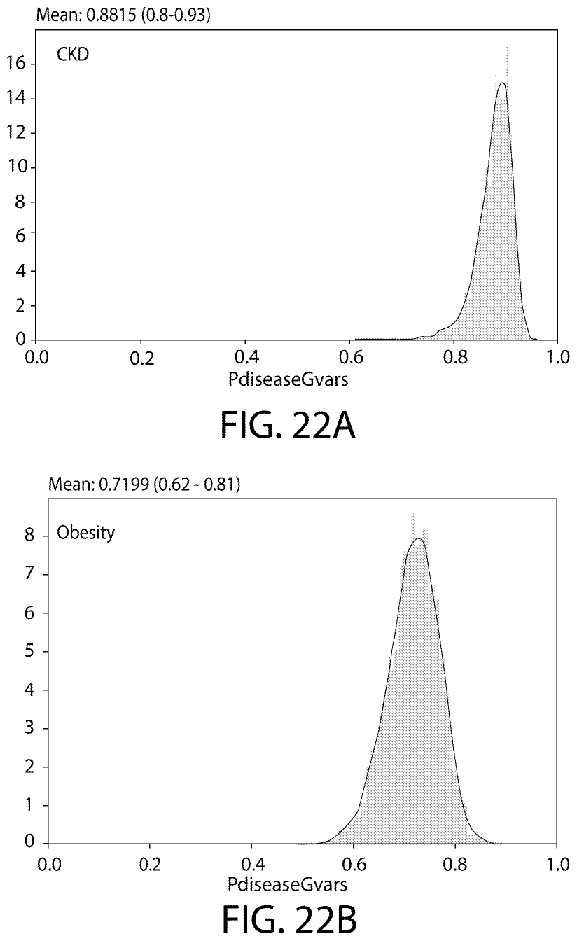

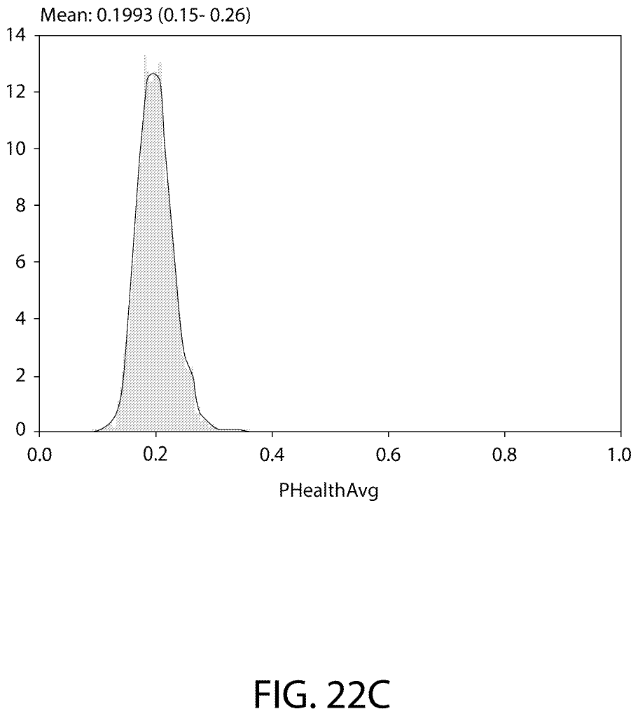

The probability of CKD was calculated from real data and three different biomarkers at levels positive for CKD. The probability of obesity from three different biomarkers at levels positive for obesity was also calculated. FIG. 22A provides a graph of the probability of CKD. FIG. 22B provides a graph of the probability of obesity. The probability of health was then calculated for each feline by subtracting the probability of organ disease from 1 and the result was simply averaged to get a distribution of probabilities for the overall pet. The distribution of probabilities for the overall feline is shown in FIG. 22C. Although this is one way in which the final health index could be calculated, there are other options including using survival analysis and/or weighting based on severity of disease.

### Example 8

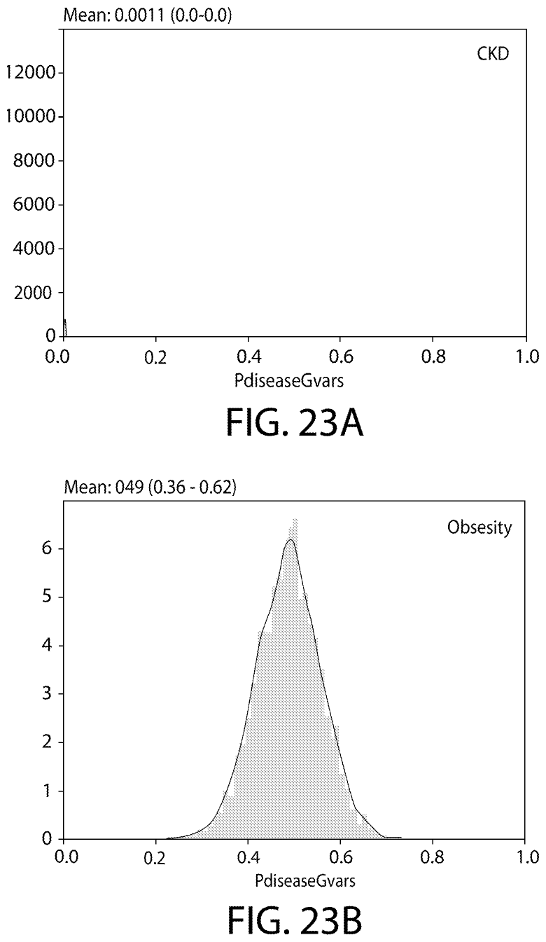

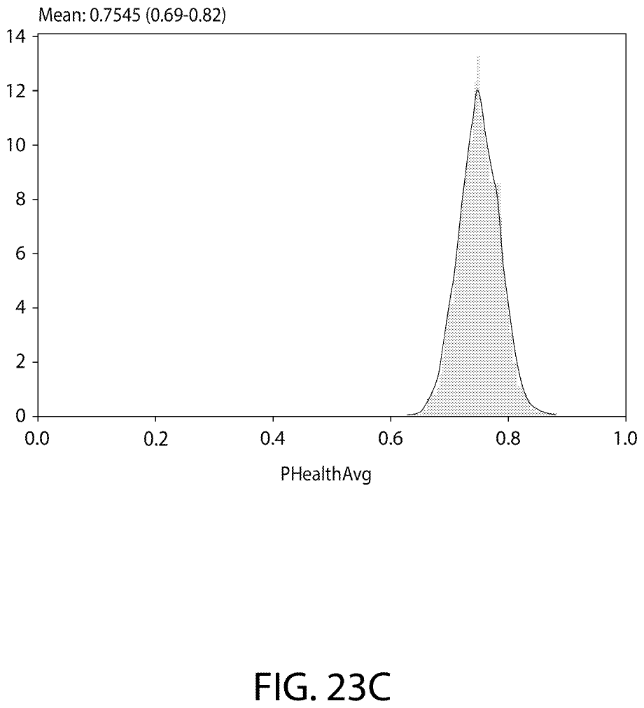

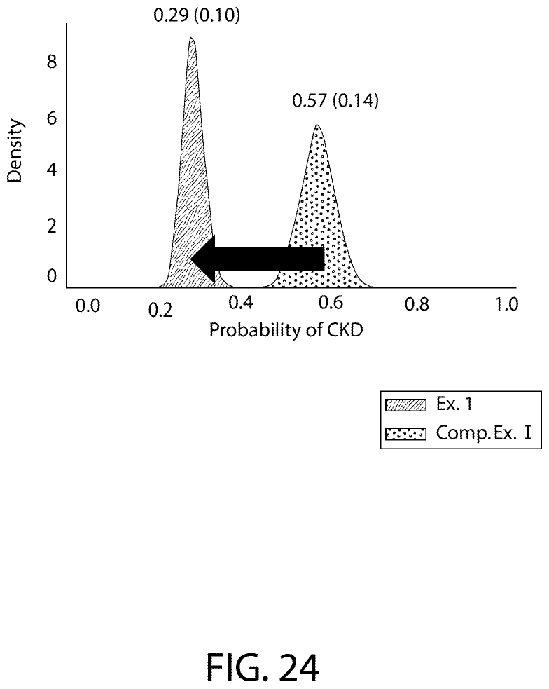

The probability of CKD was calculated from real data and three different biomarkers at levels positive for CKD with the probability of obesity from three different biomarkers at levels positive for obesity. This Example was similar to Example 7, except that the biomarkers were set to negative for the disease. FIG. 23A provides a graph of the probability of CKD; FIG. 23B provides a graph of the probability of obesity; and FIG. 23C provides a graph of the distribution of probabilities for the overall feline.

### Example 9

2400 records were created to simulate biomarkers in both ckd positive and ckd negative cats. 100 records for each age, from 1 to 20, as integers were created. Ranges for creatinine were set to match approximate ranges of data from real felines according to three age groups, with the values increasing for the older groups. Random values from those ranges were assigned to each record. SDMA for each record was calculated as a multiple of the creatinine value.

A label of 1 for ckd positive was applied to those records with values of creatinine or SDMA over a threshold value. All others were assigned 0 for ckd negative.

Noise was introduced by creating 20 records for each age, with the same range of creatinine and SDMA values regardless of age, while assigning the first 10 records as ckd positive with a value of 1 and the second 10 records were set to 0.

### Example 10

2200 records were created to represent three hypothetical diseases with associated biomarkers. Three biomarkers were assigned to each disease and thresholds to determine disease were assigned by visualizing the distributions. 100 records for each age, from 1 to 20, as integers were created. For each biomarker, the mean and standard deviation were set to create a normal distribution for each of three age groups with the mean being higher for the older ages. The appropriate distribution by age was randomly sampled for a value to be assigned to each record.

A label of 1 to represent disease presence was applied to those records whose individual biomarkers or combination of biomarkers were above the thresholds. A label of 0 was applied to the rest of the records to represent disease absence.

Noise was introduced by creating 10 records for each age, with a broader range of biomarker values that were set to 1 for disease, while another set of 10 records for each age and a broader range of biomarkers values was set to 0, for health.

### Example 11

Table 3 (below) describes the impact that an exemplary composition of the present invention can have on the Holistic Health Index/Health Index for Pets.

TABLE 3

Organ Health Score

| Organ | Before | After |
|---|---|---|
| Heart | 100 | 100 |
| Joint | 60 | 65 |
| Kidney | 80 | 80 |
| Dental | 75 | 80 |
| Skin | 60 | 95 |
| Liver | 90 | 100 |
| Digestive | 50 | 95 |
| Holistic Health Index | 74 | 88 |

As illustrated by the data in Table 3 (above), an exemplary composition of the present invention may improve the health/condition/status of various organs; and as a result, significantly and unexpectedly improve the Holistic Health Index/Health Index for Pets.

LENGTHY TABLES
The patent application contains a lengthy table section. A copy of the table is available in electronic form from the USPTO web site (https://seqdata.uspto.gov/?pageRequest=docDetail&DocID=US20250057192A1). An electronic copy of the table will also be available from the USPTO upon request and payment of the fee set forth in 37 CFR 1.19(b)(3).

What is claimed is:

1-50. (canceled)

51. A pet food composition comprising:
a component or an ingredient selected from:
a. a fiber component comprising neutral detergent fiber (NDF) and crude fiber;
b. a macronutrient component comprising a protein intact and hydrolyzed, a fat, a carbohydrate, or a combination thereof;
c. an optimized mineral component comprising controlled calcium, magnesium, phosphorus, sodium, iodine, iron, selenium, manganese, or a combination thereof;
d. an anti-oxidant component;
e. an anti-inflammatory component;
f. an osmolyte manipulator;
g. a mitochondrial co-factor;
h. an amino acid component;
i. a fatty acid component;
j. an anti-anxiety component;
k. a physiological process modulator; and
l. a combination of two or more thereof;
wherein a through l is present in an amount effective to improve HIP.

52. A pet food composition consisting essentially of:
a component or an ingredient selected from:
a. a fiber component comprising neutral detergent fiber (NDF) and crude fiber;
b. a macronutrient component comprising a protein intact and hydrolyzed, a fat, a carbohydrate, or a combination thereof;
c. an optimized mineral component;
d. an anti-oxidant component;
e. an anti-inflammatory component;
f. an osmolyte manipulator;
g. a mitochondrial co-factor;
h. an amino acid component;
i. a fatty acid component;
j. an anti-anxiety component;
k. a physiological process modulator; and
l. a combination of two or more thereof;
wherein a through l is present in an amount effective to improve HIP.

53. The pet food composition according to claim 51, wherein the pet food composition improves HIP by at least about 10%, optionally at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95%.

54. The pet food composition according to claim 51, wherein the weight ratio of NDF to crude fiber is at least about 3:2.

55. The pet food composition according to claim 51, comprising greater than about 15%, based on the total weight of the pet food composition, of NDF.

56. The pet food composition according to claim 51, comprising greater than about 10%, based on the total weight of the pet food composition, of crude fiber.

57. The pet food composition according to claim 51, wherein the NDF comprises from about 55% to about 65% of the fiber complex.

58. The pet food composition according to claim 51 wherein the crude fiber comprises from about 35% to about 45% of the fiber complex.

59. The pet food composition according to claim 51 wherein the fiber complex comprises pecan fiber, beet pulp and/or cranberry pomace.

60. The pet food composition according to claim 51 wherein the fiber complex comprises greater than about 25%, based on the total weight of the pet food composition.

61. The pet food composition according to claim 51 wherein the fiber complex is present in an amount effective to reduce circulating lipopolysaccharide.

62. The pet food composition according to claim 51, wherein the fiber complex is present in an amount effective to increase an anti-inflammatory cytokine.

63. The pet food composition according to claim 51, wherein the anti-inflammatory cytokine is selected from: interleukin-1 receptor antagonist (IL-1ra); interleukin-4 (IL-4); interleukin-6 (IL-6); interleukin-10 (IL-10); interleukin-11 (IL-11); interleukin-13 (IL-13); transforming growth factor-beta (TGF-β); and a combination of two or more thereof.

64. A pet food composition comprising:
an anti-inflammatory component;
from about 15 wt. % to about 25 wt. %, of NDF; and
from about 10 wt. % to about 20 wt. % crude fiber;
wherein the total fiber content of the pet food composition exceeds about 30 wt. %; and
wherein the pet food composition improves HIP.

65. The pet food composition according to claim 64, wherein the weight ratio of NDF to crude fiber is at least about 3:2.

66. The pet food composition according to claim 64, comprising from about 20 wt. % to about 25 wt. %, of NDF.

67. A method for treating, preventing or ameliorating a symptom associated with obesity in a companion animal comprising administering a pet food composition according to any claim 64 to a companion animal in need thereof.

68. A method for increasing pancreatic peptide and/or decreasing ghrelin in a companion animal comprising administering a pet food composition according to claim 64, to a companion animal in need thereof.

69. A method for treating, preventing, or reducing the risk of developing metabolic endotoxemia comprising administering a pet food composition according to claim 64, to a companion animal in need thereof.

70. A method for improving HIP in a companion animal comprising administering an effective amount of any one of the pet food compositions of claim 64 to a companion animal in need thereof.
